(19) United States
(12) Patent Application Publication
(10) Pub. No.: US 2024/0277245 A1
(43) Pub. Date: Aug. 22, 2024

(54) DEVICES, SYSTEMS, AND METHODS FOR REAL-TIME MONITORING OF ELECTROPHYSICAL EFFECTS DURING TISSUE TREATMENT

(71) Applicant: Virginia Tech Intellectual Properties, Inc., Blacksburg, VA (US)

(72) Inventors: Rafael V. Davalos, Aptos, CA (US); Mohammad Bonakdar, Medford, MA (US); Eduardo L. Latouche, Chelsea, MA (US); Roop L. Mahajan, Fairfax, VA (US); John L. Robertson, Floyd, VA (US); Christopher B. Arena, Blacksburg, VA (US); Michael B. Sano, Albany, NY (US)

(21) Appl. No.: 18/404,473

(22) Filed: Jan. 4, 2024

Related U.S. Application Data
(63) Continuation of application No. 16/865,031, filed on May 1, 2020, now Pat. No. 11,903,690.
Application No. 16/865,031 is a continuation of application No. 15/536,333, filed on Jun. 15, 2017, now Pat. No. 10,694,972.
(60) Provisional application No. 62/091,703, filed on Dec. 15, 2014.

(51) Int. Cl. A61B 5/0538; A61B 5/01; G01N 33/543

(52) U.S. Cl. CPC A61B 5/0538; A61B 5/01; G01N 33/5438

(57) ABSTRACT

Provided herein are devices, systems, and methods for monitoring lesion or treated area in a tissue during focal ablation or cell membrane disruption therapy.

### CROSS-REFERENCE TO RELATED APPLICATIONS

This application is a Continuation application of U.S. application Ser. No. 16/865,031, filed May 1, 2020. The '031 Application is a Continuation application of U.S. application Ser. No. 15/536,333, filed Jun. 15, 2017, which '333 Application is a National Stage application under 35 U.S.C. § 371 of International Application No. PCT/US2015/065792, filed Dec. 15, 2015, which international application claims the benefit of U.S. Provisional Application Ser. No. 62/091,703 filed on Dec. 15, 2014, each of which are incorporated herein in their entireties.

### STATEMENT REGARDING FEDERALLY SPONSORED RESEARCH OR DEVELOPMENT

This invention was made with government support under grant number IIP-1346343 awarded by the National Science Foundation. The government has certain rights in the invention.

### BACKGROUND

Focal ablation and other cell membrane disruption therapies and molecule delivery mechanisms are used in many clinical and research applications. As such, monitoring techniques for lesion/treatment area are desirable. As such, there exists a need for improved monitoring techniques for use, inter alia, focal ablation and other cell membrane disruption therapies.

### SUMMARY

Provided herein are embodiments of an electrical conductivity sensor having an impedance sensor, where the impedance sensor can be configured to measure a low-frequency and a high-frequency impedance and a substrate, where the impedance sensor is coupled to the substrate. The substrate can be flexible. In embodiments, the impedance sensor can contain two or more electrical conductors. The electrical conductors can be in a bipolar configuration. The electrical conductors can be in a tetrapolar configuration. In embodiments, the electrical conductivity sensor can have two impedance sensors that can be coupled to the substrate such that they are orthogonal to each other.

In embodiments, the electrical conductivity sensor can have more than one impedance sensor. In some embodiments, the impedance sensors can be configured in an array. In embodiments having more than one impedance sensor, the electrical conductivity sensor can further contain a common ground, where each impedance sensor is coupled to the common ground. In embodiments having more than one impedance sensor, the electrical conductivity sensor can further contain a common counter electrode, wherein the common counter electrode can be coupled to the substrate.

In embodiments, the impedance sensor(s) can have interdigitated electrodes. In embodiments, the impedance sensor(s) can further contain a receptor molecule configured to specifically bind a target molecule, wherein the receptor molecule is coupled to the sensor(s).

In embodiments, the electrical conductivity sensor can contain one or more sensors configured to detect a tissue characteristic selected from the group of: pH, temperature, a chemical concentration, a nucleic acid concentration, a gas amount, or combinations thereof.

Also provided herein are embodiments of an electrical conductivity probe having an elongated member and an electrical conductivity sensor as described herein where the electrical conductivity sensor can be coupled to the elongated member. In embodiments, the electrical conductivity sensor can be removably coupled to the elongated member.

Also provided herein are embodiments of a system having an electrical conductivity probe as described herein, a treatment probe configured to deliver an energy to a tissue, where the energy can be sufficient to disrupt a cell membrane, an impedance analyzer, where the impedance analyzer can be coupled to the electrical conductivity probe, a low voltage power supply, where the low voltage power supply can be coupled to the electrical conductivity probe and can be configured to deliver a low voltage energy to the electrical conductivity probe, a waveform generator, where the waveform generator can be coupled to the low voltage power supply, a gate driver, where the gate driver can be coupled to the waveform generator and the low voltage power supply, a high voltage switch, where the high voltage switch can be coupled to the treatment probe and the impedance analyzer; and a high voltage power supply, where the high voltage power supply can be coupled to the high voltage switch.

In embodiments, the system can further contain a computer. The computer can be coupled to the impedance analyzer and the computer can contain processing logic that can be configured to determine the position of lesion or treated area front within a tissue undergoing focal ablation/cell membrane disruption therapy. The processing logic can be further configured to generate a signal to a user when the position of lesion or treated area front has reached a predetermined position within the tissue. The processing logic can be configured to automatically manipulate the system to adjust or stop treatment of a tissue by the treatment probe when the position of lesion or treated area front has reached a predetermined position within the tissue.

In embodiments, the treatment probe and the electrical conductivity probe can be the same probe. In embodiments, the treatment probe and the electrical conductivity probe are separate probes. The treatment probe can be coupled to a grounding pad located elsewhere relative to the treatment probe in or on the body of a subject being treated.

Also provided herein are embodiments of a method of monitoring the lesion or treated area front or size during focal ablation or cell membrane disruption therapy, the method have the steps of inserting an electrical conductivity probe as described herein into a tissue, inserting a treatment probe into the tissue, applying a treatment to the tissue, wherein the treatment comprises applying an energy to the tissue via the treatment probe, and measuring a characteristic of the tissue continuously during treatment, determining if there is a change in the tissue characteristic. The characteristic can be impedance. In some embodiments, the step of measuring can include measuring both low-frequency impedance and high-frequency impedance and further comprising the step of stopping or adjusting treatment when low-frequency impedance is equal to high-frequency impedance. In embodiments, the characteristic can be pH, temperature, a gas concentration, a chemical concentration, a nucleic acid concentration, or a combination thereof. In some embodiments, the method can contain the step of stopping or adjusting a treatment when a change in the tissue characteristic is detected. In embodiments, the method can contain the step of alerting a user when a change in the tissue characteristic is detected.

In some embodiments, where the electrical conductivity probe includes an impedance sensor array, the method can include the step of determining the location of the lesion or treated area front or size by comparing impedance data between two or more impedance sensors of the impedance sensor array. In embodiments, the method can include the step of comparing the lesion or treated area front or size to a threshold value and stopping treatment when lesion or treated area front or size is greater than or equal to the threshold value. In embodiments, the method can include the step of comparing the lesion or treated area front or size to a threshold value and alerting a user when lesion or treated area front or size is greater than or equal to the threshold value.

The method can include the steps of comparing measured changes in impedance to a solution for the electric field distribution during focal ablation or cell membrane disruption and determining the 2D/3D lesion or treated area geometry of the lesion or treated area volume. In embodiments, the method can include the step of overlaying the 2D/3D lesion or treated area geometry on one or more medical images of a subject to generate an image overlay. The method can include the step of visualizing lesion or treatment area front migration or lesion or treatment area growth from the image overlay.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows embodiments of an electrical conductivity sensor.

FIG. 2 shows embodiments of an electrical conductivity sensor.

FIG. 3 shows embodiments of an electrical conductivity sensor.

FIG. 4 shows embodiments of an electrical conductivity sensor.

FIG. 5 shows embodiments of an electrical conductivity sensor.

FIG. 6 shows embodiments of an electrical conductivity sensor.

FIG. 7 shows embodiments of an electrical conductivity sensor.

FIG. 8 shows embodiments of an electrical conductivity sensor.

FIG. 9 shows embodiments of an electrical conductivity probe.

FIG. 10 shows embodiments of an electrical conductivity probe.

FIG. 11 shows embodiments of an electrical conductivity probe.

FIG. 12 shows embodiments of an electrical conductivity probe.

FIG. 13 shows embodiments of an electrical conductivity probe.

FIG. 14 shows embodiments of a system configured to monitor lesion/treated area formation in real-time.

FIG. 15 shows embodiments of a system configured to monitor lesion/treated area formation in real-time.

FIGS. 16A-16B show embodiments of operation of an electrical conductivity probe during treatment to monitor lesion/treated area formation in real-time.

FIGS. 17A-17C show embodiments of operation of an electrical conductivity probe during treatment to monitor lesion/treated area formation in real-time.

FIG. 18 shows an image of an embodiment of an electrical conductivity sensor.

FIG. 19 shows an image of an embodiment of an electrical conductivity sensor.

FIG. 20 shows an image of an embodiment of an electrical conductivity sensor.

FIG. 21 shows an image of an embodiment of an electrical conductivity probe.

FIGS. 22A-22J shows steps in a process for manufacturing an electrical conductivity sensor.

FIGS. 23A-23B show a three dimensional finite element model to simulate IRE treatment of liver tissue with two needle electrodes.

FIGS. 24A-24B show the simulated electrical conductivity in the tissue resulted from IRE (FIG. 24A) and simulated extrapolation of point specific measurements in three dimensions to determine the spatial-temporal conductivity map and electric field distribution (FIG. 24B).

FIGS. 25A and 25B show images of a probe FIG. 25A and placement within a sample of porcine liver. The dashed circle in FIG. 25B indicates the treated area. The black dots indicate location of the sensors of the probe within the tissue.

FIG. 26 shows a graph demonstrating tissue resistance (ohms) after delivery of a series of high-frequency irreversible electroporation (HFIRE) pulses to the porcine liver of FIG. 25B as measured by the probe of FIG. 25A.

FIG. 27 shows a graph demonstrating % change in tissue resistance between varying sensors after delivery of a series of high-frequency irreversible electroporation (HFIRE) pulses to the porcine liver of FIG. 25B as measured by the probe of FIG. 25A.

FIGS. 28A-28D show images of a 3D isometric view of the probe onto ortho-planes from stacked CT images of patient anatomy.

FIGS. 29A-29C show graphs demonstrating finite element modeling (FEM) of electric field magnitude along the length of the probe in a potato model, where N=10 (FIG. 29A), N=30 (FIG. 29B), and N=100 (FIG. 29C).

FIGS. 30A-30C show graphs demonstrating experimental results of conductivity change as measured by different sensor pairs along the length of the probe in a potato model, where N=10 (FIG. 30A), N=30 (FIG. 30B), and N=100 (FIG. 30C).

FIGS. 31A-31C show photos demonstrating experimental ablations after delivering a series of IRE pulses to a potato model where N=10 (FIG. 31A), N=30 (FIG. 31B), and N=100 (FIG. 31C).

FIG. 32 shows embodiments of installation of a sensor array on a probe and the electrical connections to the sensor.

FIGS. 33A-33B shows the electrical impedance spectrum of the porcine liver (FIG. 33A) along with the equivalent circuit model of the tissue (FIG. 33B).

FIG. 34 shows an embodiment of a system where a monopolar electrode and a grounding pad are used to deliver the high voltage pulses.

FIG. 35 shows another embodiment of a system where a monopolar electrode and a grounding pad are used to deliver the high voltage pulses.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of mechanical engineering, electrical engineering, physiology, medical science, veterinary science, bioengineering, biomechanical engineering, physics, and the like, which are within the skill of the art. Such techniques are explained fully in the literature.

### Definitions

As used herein, “about,” “approximately,” and the like, when used in connection with a numerical variable, generally refers to the value of the variable and to all values of the variable that are within the experimental error (e.g., within the 95% confidence interval for the mean) or within ±0.10% of the indicated value, whichever is greater.

As used herein, “control” is an alternative subject or sample used in an experiment for comparison purposes and included to minimize or distinguish the effect of variables other than an independent variable. A “control” can be a positive control, a negative control, or an assay or reaction control (an internal control to an assay or reaction included to confirm that the assay was functional). In some instances, the positive or negative control can also be the assay or reaction control.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “biocompatible” or “biocompatibility” refers to the ability of a material to be used by a patient without eliciting an adverse or otherwise inappropriate host response in the patient to the material or an active derivative thereof, such as a metabolite, as compared to the host response in a normal or control patient.

As used herein, “therapeutic” can refer to curing and/or treating a symptom of a disease or condition.

The term “treating”, as used herein, can include inhibiting and/or resolving the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The term “preventing”, as used herein includes preventing a disease, disorder or condition from occurring in a subject, which can be predisposed to the disease, disorder and/or condition but has not yet been diagnosed as having it. As used herein, “preventative” can refer to hindering or stopping a disease or condition before it occurs or while the disease or condition is still in the sub-clinical phase.

The term “target molecule” can refer to any specific desired molecule including, but not limited to, a nucleic acid, oligonucleotide, polynucleotide, peptide, polypeptide, chemical compound, or other molecule that can specifically bind to a receptor molecule. Typically, the target molecule refers to a molecule that can be located in a sample or tissue whose presence and/or amount can be determined by detecting its binding to known receptor molecule.

The term “receptor molecule” can refer to a molecule that can specifically bind to a target molecule. A receptor molecule can be a nucleic acid, oligonucleotide, polynucleotide, peptide, polypeptide, chemical compound, or other molecule. Receptor molecules can be, for example, antibodies or fragments thereof or aptamers. The receptor molecule can be bound, fixed, or otherwise attached to a surface, sometimes in known location (e.g. as in an array), and can be exposed to a sample such that if a target molecule is present, the target molecule can interact and specifically bind with the receptor molecule. The specific binding can, in some cases, trigger a signal that can provide quantitative and/or qualitative information regarding the target molecule.

As used herein, “specific binding,” “specifically bound,” and the like, refer to binding that occurs between such paired species as nucleotide/nucleotide, enzyme/substrate, receptor/agonist, antibody/antigen, and lectin/carbohydrate that can be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, “aptamer” refers to single-stranded DNA or RNA molecules that can bind to pre-selected targets including proteins with high affinity and specificity. Their specificity and characteristics are not directly determined by their primary sequence, but instead by their tertiary structure.

### Discussion

Focal cell ablation and focal cell membrane disruption techniques can be used to selectively destroy undesired tissue, deliver drugs to cells and tissues, and deliver nucleic acids to cells. Focal ablation and membrane disruption techniques can be thermally or non-thermally based. Thermally based techniques use heat to ablate cells or disrupt cell membranes and include, but are not limited to, radiofrequency (RF) ablation, laser ablation, cryo-ablation, and ultrasound. Other thermal focal ablation/membrane disruption techniques will be appreciated by those of ordinary skill in the art. Non-thermal techniques can rely on the generation or application of an electric field to cells to disrupt (reversibly or irreversibly) the cell membrane, which increases the permeability or kills the cells. Non-thermal focal ablation/membrane disruption techniques include, but are not limited to electroporation. Other Non-thermal focal ablation/membrane disruption techniques will be appreciated by those of ordinary skill in the art. During these techniques, it is difficult to determine the extent of treatment within a tissue being treated. As such, current procedures relying on focal ablation and membrane disruption techniques are imprecise, which can result in undesirable side effects, destruction of, or gene/transcript/protein modification in normal or otherwise healthy cells.

Membrane permeability changes induced by focal ablation/cell membrane disruption techniques at the cell level can translate into changes in impedance at the tissue level. Known devices and methods of monitoring tissue impedance, such as during electroporation, have several drawbacks. Primarily, they rely on bulk tissue properties as opposed to measurements at well-defined points within the tissue being treated. Bulk changes can be useful in describing how the dielectric properties of the tissue change as a whole during treatment. However, there is no specificity in terms of the location where treatment is occurring. In known devices and methods, this information is usually inferred from correlations with predications of the electric field distribution in the tissue. In other words, the treatment zone is defined as the area above a pre-determined threshold that is based on the inferred correlations and predications. The bulk measurements can be made either through the treatment electrodes or with a separate set of electrodes, where the electrodes located in proximity to each other.

As an alternative, electrical impedance tomography (EIT) can be used to map the tissue dielectric potential throughout the entire treatment region based on solutions to a nonlinear inverse that accounts for surface electrical measurements. However, this imaging technique is complicated by the required placement of an electrode array around the periphery of the target tissue. Placement of the electrode array can be difficult to implement clinically because some tumors and other target tissues do not accommodate the placement of such an array due to geometrical/anatomical constraints or the presence of highly insulating anatomical structures such as the skull or skin. Further, EIT suffers from the limitations associated with the resolution of reconstructed images, which relies heavily on the accurate placement and number of external electrodes. Moreover, none of the existing technologies and methods can achieve active, real-time monitoring of the lesion or treated area front during focal ablation and cell membrane disruption procedures.

With these shortcomings in mind, described herein are devices and systems that can be configured to monitor a lesion or treated area front in real-time during focal ablation/membrane disruption therapy. The devices and systems can be configured with a sensor array to detect a lesion or treated area front. The devices and systems provided herein can be used to actively monitor focal ablation/cell membrane disruption therapy in real-time and thus can allow a practitioner to control, adjust, and/or discontinue treatment in response to front migration to minimize treatment side effects.

Also described herein are methods of monitoring a lesion or treated area front in real-time in tissue during focal ablation/membrane disruption. The methods can include alerting a user when the front has reached a desired location. The methods can utilize both low- and high-frequency electrical impedance measurements to determine if the tissue area surrounding a sensor has been ablated or treated. The devices, systems and methods described herein can provide for focal ablation/membrane disruption techniques and therapies with improved specificity than current techniques and devices. Other devices, systems, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Systems and Devices for Real-Time Impedance Monitoring

During focal ablation or cell membrane disruption procedures, as the procedure continues the treated area or lesion expands out from the treatment source. A feature common to these types of therapies is a change in the membrane permeability of the cell membranes that have been stimulated during focal ablation or cell membrane disruption. Focal ablation and other membrane disruption techniques can result in a change in impedance in due to a change in the permeability of the cells that have been sufficiently stimulated during focal ablation or cell membrane disruption.

As the lesion or treated area forms as treatment continues, an increasing number of cells in the tissue surrounding the treatment source undergo a membrane disruption and thus a change in the impedance of the cells in that area. As the lesion/treated area grows, a front can be formed that forms a boundary between treated and untreated cells. The treated cells and the untreated cells can have different impedances or other characteristics (e.g. pH and temperature). By measuring the impedance or other characteristic between two or more points in the tissue during treatment, it can be possible to determine if the front lies between those two points. The position of the lesion/treated area front within a tissue being treated can also be made by measuring impedance or other tissue characteristic at a single point and comparing that to a base line or prior measurement from that point.

Provided herein are systems and devices that can be configured to detect and determine the location of a lesion/treated area front in real-time during a focal ablation or cell membrane disruption therapy. The systems and devices can also be configured to generate 3D images and models from lesion/treated area front measurements that can provide the volume of a lesion/treated area. The systems and devices can be configured to provide automatic control of a treatment in response to detection of the migration of the lesion/treated area front. The systems and devices can be configured to provide a signal to a user in response to detection of the migration of the lesion/treated area front.

Biological tissue is a combination of extracellular space, cellular membranes, and subcellular structures, each of which contains organic molecules and ions in different structural arrangements. This can result in a broad spectrum of dielectric properties across multiple frequencies. In other words, the dielectric properties of tissue are frequency dependent. From around 0.1 Hz to 10 MHz, there exist two main dispersive regions: (1) the α, or low frequency, dispersion region and (2) the β, or high frequency, region. The α region ranges from about 0.1 Hz to about 10 Hz and the β region ranges from about 0.1 MHz to about 10 MHz. The α region is due to counter ion polarization effects along cell membranes. The β region is due to the Maxwell-Wagner effects. This describes the charging and relaxation effects along cell membranes, which act as barriers to the movement of ions.

Above the β dispersion, cell membranes have negligible impedance and current can pass freely through the cell membrane. This is similar to what happens during, for example, electroporation, when pore formation reduces the membrane impedance and permits current to enter the cell. As a result, low frequency (a region) electrical measurements at a location in a tissue before and after focal ablation or cell membrane disruption can be compared to determine if the focal ablation or cell membrane disruption has reached its endpoint at that position in the tissue. At the endpoint, the low frequency (a region) impedance is about equal to the high-frequency (β region) impedance, which is due to the focal ablation or cell membrane disruption in that region of the tissue. Stated differently, in a formed lesion or treated area, the low frequency (a region) impedance is about equal to the high-frequency (R region) impedance. Thus, comparison of the low frequency (a region) impedance and the high-frequency (β region) impedance can be used to determine lesion formation in that area of tissue due to focal ablation/cell membrane disruption treatment.

In some embodiments, the systems and devices can be configured to detect a focal ablation or cell membrane disruption in treatment area by simultaneously measuring both a region and β region impedance in a tissue. The systems and devices described herein can be configured to monitor, in real-time, the size of a treated area during a focal ablation or cell membrane disruption procedure. The devices and systems can contain an electrical conductivity sensor, which can contain an impedance sensor or impedance sensor array. The electrical conductivity sensor can be configured to measure both low-frequency (a region) impedance and high-frequency (β region) impedance. The electrical conductivity sensor can be integrated with or operatively coupled to an electrical conductivity probe and/or be integrated with or operatively coupled to a treatment probe. In embodiments, the frequency is between 0.001 and 100 GHZ, preferably between 1 kHz and 1 MHZ.

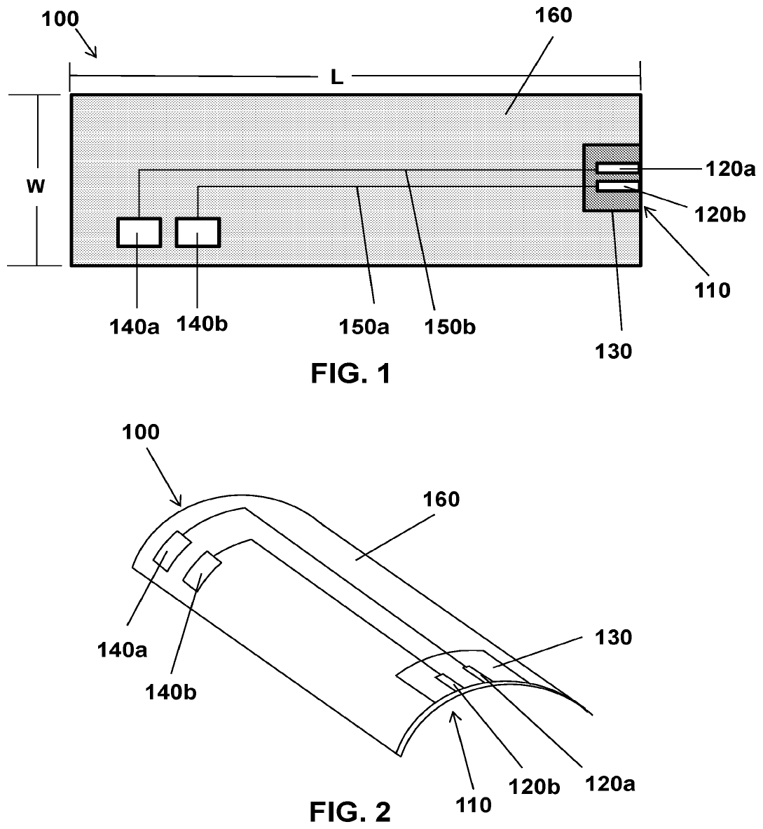

Electrical Conductivity Sensors With a general description in mind, attention is directed to FIGS. 1-8, which show embodiments of electrical conductivity sensors that can be configured to measure tissue impedance, a change in tissue impedance between points in a tissue, migration of a lesion/treated area front, and/or both low-frequency (a region) impedance and high-frequency (β region) impedance.

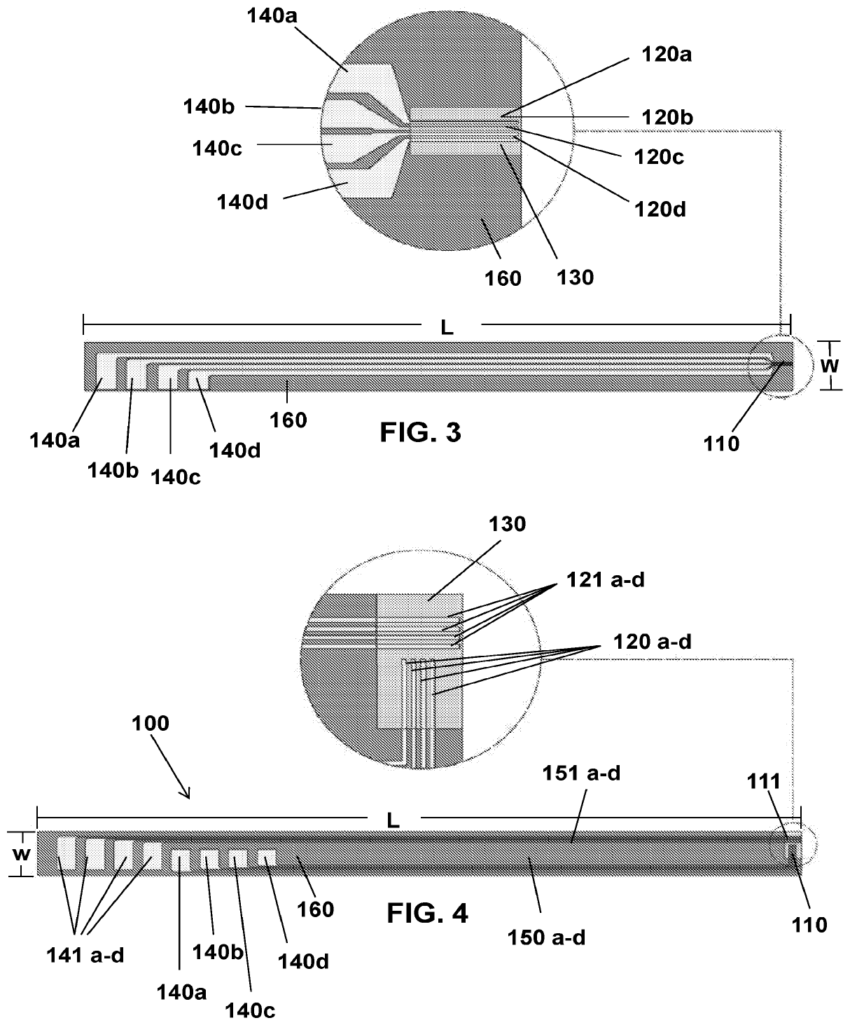

Discussion begins with FIG. 1, which shows one embodiment of an electrical conductivity sensor 100 that can be configured to measure a change in tissue impedance between points in a of tissue, and/or both low-frequency (a region) impedance and high-frequency (β region) impedance. The electrical conductivity sensor 100 can have an impedance sensor 110 at least two electrical conductors 120 a,b (collectively 110). In some embodiments, the impedance sensor 110 can have an even number of electrical conductors 120. In some embodiments the impedance sensor 110 can have 2, 3, 4, 5, 6, 7, 8, 9, 10 or more electrical conductors 120. In some embodiments, the impedance sensor 110 can be configured to measure impedance using a bipolar configuration of electrodes 120 (see e.g. FIG. 1). In other embodiments, the impedance sensor 110 can be configured to measure impedance using a tetrapolar configuration (see e.g. FIG. 3). It will be appreciated that the sensor electrodes, in any given configuration, can be separate from any source and sink electrodes that can be used for delivering the focal ablation/cell membrane disruption therapy.

The electrical conductors 120 can be coupled to bonding pads 140 a,b (collectively 140). In some embodiments, each electrical conductor 120 is coupled to an individual bonding pad 140. The electrical conductors 120 can be coupled to the bonding pad(s) 140 via electrical leads 150 a,b (collectively 150). The electrical conductor 120, the bonding pad(s) 140, and the lead(s) 150 can be coupled to a substrate 160. In some embodiments, the electrical conductors 120 can be coupled to an impedance sensor substrate 130. The impedance sensor substrate 130 can be coupled to the substrate 160. In some embodiments, the electrical conductors 120 can be attached directly to the substrate 160. The electrical conductivity sensor 100 can be configured such that at least a portion of one or more of the electrodes is exposed to the tissue when in use.

The electrical conductivity sensor 100 can have a length (l), a width (w), and a thickness. The length can range from about 1 mm to 1000 mm or more. The width can range from about 0.1 mm to about 50 mm or more. The thickness can range from about 0.1 micron to about 1000 microns or more.

As shown in FIG. 2, the electrical conductivity sensor 100 can be flexible. The substrate 160 and the optional impedance sensor substrate 130 can be made out of any suitable material. The material can be biocompatible. Suitable materials include, but are not limited to ceramics (porcelain, alumina, hydroxyapatite, zirconia), polymers (e.g. thermoplastic elastomers (e.g. silicone elastomers, styrene block copolymers, thermoplastic copolyesters, thermoplastic polyesters, thermoplastic polyamides, thermoplastic polyolefins, thermoplastic polyurethanes, thermoplastic vulcanizates), polyvinyl chloride, fluoropolymers (PTFE, modified PTFE, FEP, ETE, PFA, MFA), polyurethane, polycarbonate, silicone, acrylics, polypropylene, low density polyethylenes, nylon, sulfone resins, high density polyethylenes, natural polymers (cellulose, rubber, alginates, carrageenan), polyimide, polyether ether ketone), metals (e.g. gold, silver, titanium, platinum), metal alloys (e.g. stainless steel, cobalt alloys, titanium alloys), glass, and combinations thereof.

The leads 150, bonding pads 140 and electrical conductors 120 can be made of a suitable conductive or semi-conductive material. The material can be flexible. The materials can be biocompatible. Suitable conductive and semi-conductive materials include, without limitation, gold, silver, copper, aluminum, nickel, platinum, palladium, zinc, molybdenum, tungsten, graphite, Indium tin oxide, conductive organic polymers (e.g. polyacetylene, polyphenylene vinylene, polypyrrole, polythiophene, polyaniline, and polyphenylene sulfide), silicon, germanium, cadmium, indium, and combinations thereof.

In operation, a known electrical current can be passed through at least one of the electrical conductors 120. A voltage is then induced in at least one of the other electrical conductors 120 As such, in embodiments, where there are only two electrical conductors 120 (a bipolar configuration) (see e.g. FIG. 1), a known current can be passed through one electrical conductor (120a) and a voltage is then induced in the other electrical conductor (120b). As shown in FIG. 3, where there are more than two electrical conductors 120 a-d (e.g. a tetrapolar configuration), a current can be passed through the outer most electrical conductors 120 a,d and the induced voltage across the inner electrical conductors 120 b,c can be measured. Other suitable configurations will be appreciated by those of skill in the art. In any embodiment, the high-frequency and low frequency impedance can be measured from the induced voltages. As described elsewhere herein, the high-frequency and low-frequency impedance can be used to determine if a particular region of tissue has been treated and/or the area and/or volume of tissue that has been effectively treated.

Some tissues have anisotropic electrical properties, which can be due to the directional growth of the cell. As such, in some instances it is desirable to measure the electrical conductivities in two orthogonal directions. With this in mind, attention is directed to FIG. 4, which shows an embodiment of an electrical conductivity sensor configured to measure both high- and low-frequency impedance in two orthogonal directions.

As shown in FIG. 4, the electrical conductivity sensor can have at least two impedance sensors 110 and 111. The first impedance sensor 110 can have a first set of electrical conductors 120 a-d. The second impedance sensor 111 can have a second set of electrical conductors 121 a-d. The first 120 and second 121 sets of electrical conductors can be coupled to a substrate 160 and/or impedance sensor substrate 130 such that the first set of electrical conductors 120 and the second set of electrical conductors 121 are orthogonal to each other. In this way, the first 110 and the second 111 impedance sensors can be said to be orthogonal to each other in these embodiments.

While FIG. 4 shows the impedance sensors 110, 111 in a tetrapolar configuration it will be appreciated by those of skill in the art that they can be configured in any suitable manner, for example, as previously described with respect to FIGS. 1-3. Likewise, each impedance sensor 110, 111 can have at least two electrical conductors 120, 121. In some embodiments, each impedance sensor 110, 111 can have 3, 4, 5, 6, 7, 8, 9, 10 or more electrical conductors. The impedance sensors 110, 111 can have the same number or a different number of electrical conductors 120, 121 as each other. The dimensions of these embodiments of the electrical conductivity sensor 100 can be as described with respect to FIGS. 1-3 above. The electrical conductivity sensor 100 and components thereof can be made from suitable materials as previously described with respect to FIGS. 1-3. As previously described, each electrical conductor 120, 121, can be coupled to a bonding pad 140 a-d and 141 a-d via an electrical leads 150 a-d and 151 a-d. The operation of each set of electrodes 120, 121 to measure impedance can be as described with respect to FIGS. 1-3 above.

FIGS. 1-4 demonstrate embodiments of an electrical conductivity sensor 100 that contain electrical conductors at a single location on the electrical conductivity sensor 100. As described elsewhere herein it can be desirable to measure the size of a treatment area in a tissue during focal ablation/cell membrane disruption therapy. During therapy, the lesion formed will grow in size, and as such, it can be desirable to measure this growth without the need for repositioning the electrical conductivity sensor, or probe that it can be coupled to, during treatment.

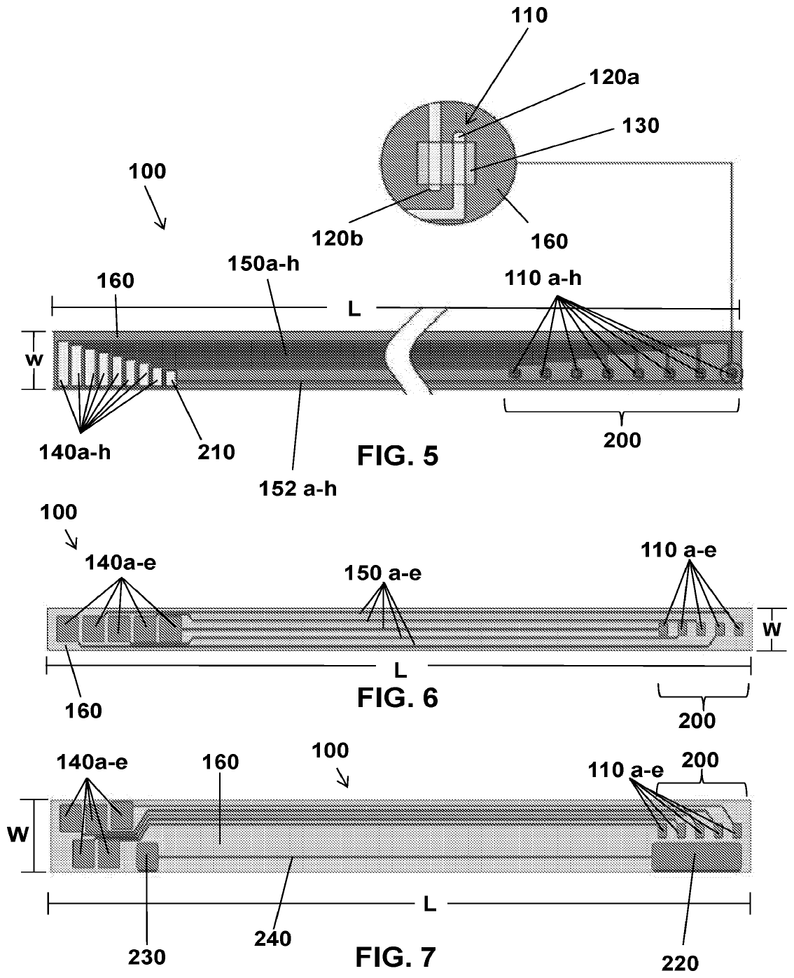

With this in mind, attention is directed to FIGS. 5-8 which show embodiments of an electrical conductivity sensor 100 that has a sensor array. The electrical conductivity sensor 100 having a sensor array can be configured to measure impedance. In some embodiments, the electrical conductivity sensor 100 having a sensor array 200 can be configured to detect both high- and low-frequency impedance having an impedance sensor array 200. In some embodiments the sensor array 200 can be configured to detect another tissue characteristic, including but not limited to, pH, temperature, drug concentration, chemical concentration, gas concentration and combinations thereof. As such, in some embodiments, the lesion/treated area front can be determined by measuring these characteristics.

Discussion continues with FIG. 5 which shows one embodiment of an electrical conductivity sensor 100 having an impedance sensor array 200. In the embodiments depicted by FIG. 5, the impedance sensor array 200 has at least two impedance sensors 110 a-h. While FIG. 5 shows an impedance sensor array 200 having eight (8) impedance sensors 110, it will be appreciated that the impedance sensor array 200 can have 2, 3, 4, 5, 6, 7, 8, 9, 10, 11, 12, 13, 14, 15, 16, 17, 18, 19, 20 or more impedance sensors 110. Each impedance sensor 110 can be coupled to a bonding pad 140 a-h and a common ground 210 via electrical leads 150 a-h and 152 a-h. While FIG. 5 shows an impedance sensor array 200 having eight (8) bonding pads 140, it will be appreciated that the impedance sensor array 200 can have 2, 3, 4, 5, 6, 7, 8, 9, 10, 11, 12, 13, 14, 15, 16, 17, 18, 19, 20 or more bonding pads 140. The dimensions of these embodiments of the electrical conductivity sensor 100 can be as described with respect to FIGS. 1-3 above. The electrical conductivity sensor 100 and components thereof can be made from suitable materials as previously described with respect to FIGS. 1-3. In some embodiments, the electrical conductivity sensor 100 having an impedance sensor array 200 can contain include two current injection electrodes on either end of the electrode array.

Measurement of low-frequency and/or high-frequency impedance of each impedance sensor 110 of the impedance sensor array 200 can be as previously described with respect to FIGS. 1-3. Further, differences in impedance measurements between two or more different impedance sensors 110 of the impedance sensor array 200 can be determined. In this way it is possible to determine the extent of the lesion formed by focal ablation/cell membrane disruption therapy. Stated differently, the change in the electrical impedance of different combinations of impedance sensors 110 of the impedance sensor array 200 can be evaluated and the lesion size, and/or lesion/treated area front can be determined based on the impedance or other tissue characteristic measurements evaluated. This is discussed in greater detail elsewhere herein.

In some embodiments, the sensors 110 can be functionalized with one or more receptor molecules configured to specifically bind a target molecule. This can make the impedance measurement more selective toward identification of certain intracellular substances, including proteins and ions that are released during electroporation. This modification can enhance the capability of the sensor to detect the lesion front.

FIG. 6 shows another embodiment of an electrical conductivity sensor 100 having an impedance sensor array 200. The impedance sensor array 200 has at least two impedance sensors 110 a-e. While FIG. 6 shows an impedance sensor array 200 having five (5) impedance sensors 110, it will be appreciated that the impedance sensor array 200 can have 2, 3, 4, 5, 6, 7, 8, 9, 10, 11, 12, 13, 14, 15, 16, 17, 18, 19, 20 or more impedance sensors 110. Each impedance sensor 110 can be coupled to a bonding pad 140 a-e via electrical leads 150 a-e. While FIG. 6 shows an impedance sensor array 200 having five (5) bonding pads 140, it will be appreciated that the impedance sensor array 200 can have 2, 3, 4, 5, 6, 7, 8, 9, 10, 11, 12, 13, 14, 15, 16, 17, 18, 19, 20 or more bonding pads 140. The electrical impedance measured by any combination of impedance sensors can be determined and correlated to the lesion size. The dimensions of these embodiments of the electrical conductivity sensor 100 can be as described with respect to FIGS. 1-3 above. The electrical conductivity sensor 100 and components thereof can be made from suitable materials as previously described with respect to FIGS. 1-3.

FIG. 7 shows further embodiments of an electrical conductivity sensor 100 having an impedance sensor array 200. These embodiments are the same as those described in relation to FIG. 6 except that they further contain a common counter electrode 220. The common counter electrode 220 can be coupled to the substrate 160. The common counter electrode 220 can be coupled to a bonding pad 230 via an electrical lead 240, which both can also be coupled to the substrate 160. In operation, all impedances measured by the impedance sensors 110 of the impedance sensor array 220 can be measured with respect to the common counter electrode 220. It will be appreciated that a common counter electrode can also be used in embodiments described in FIG. 5. The dimensions of these embodiments of the electrical conductivity sensor 100 can be as described with respect to FIGS. 1-3 above. The electrical conductivity sensor 100 and components thereof can be made from suitable materials as previously described with respect to FIGS. 1-3.

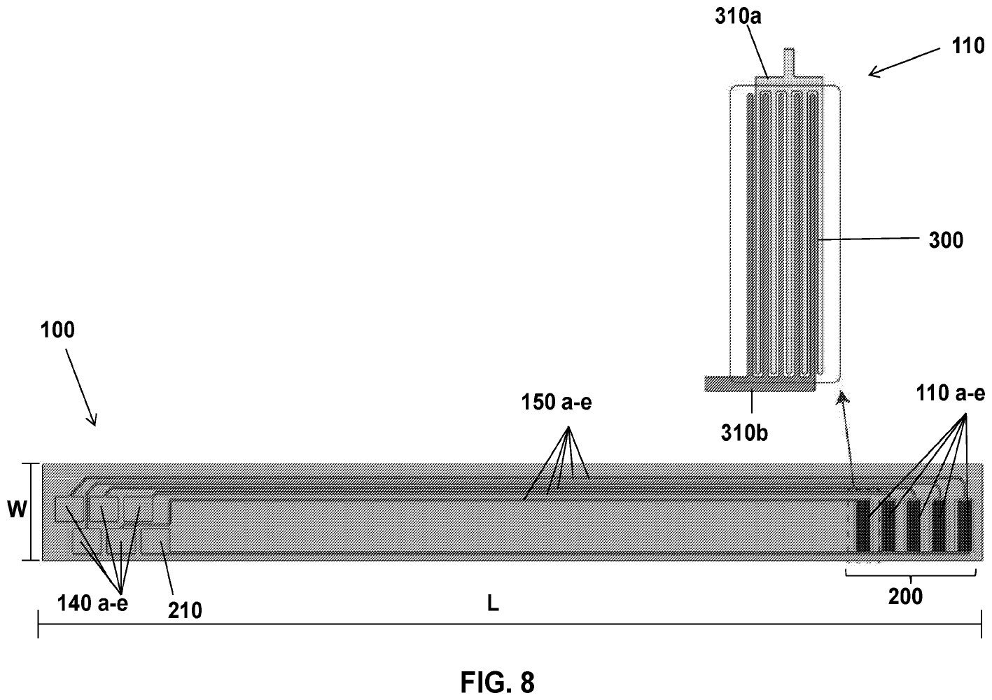

FIG. 8 shows further embodiments of an electrical conductivity sensor 100 having an impedance sensor array 200. The impedance sensor array 200 can contain impedance sensors 110 having interdigitated electrodes 300. In embodiments, the impedance sensor can have a pair of electrode sets 310 a,b (collectively 310), where each electrode set has an even number of electrodes (e.g. 2, 4, 6, 8, 10 etc.) and can be interdigitated with each other as shown in FIG. 8. This interdigitated configuration can increase the sensitivity of the impedance sensor 110. While not being bound to theory, it is believed that the increase in sensitivity can be attributed to the increased current density across the interdigitated pair of electrode sets 310 relative to a non-interdigitated electrode set.

While FIG. 8 shows an impedance sensor array 200 having five (5) impedance sensors 110, it will be appreciated that the impedance sensor array 200 can have 2, 3, 4, 5, 6, 7, 8, 9, 10, 11, 12, 13, 14, 15, 16, 17, 18, 19, 20 or more impedance sensors 110. The impedance sensors 110 can be coupled to a substrate 160 as previously described in relation to e.g. FIGS. 5-7. Each impedance sensor 110 can be coupled to a bonding pad 140 via electrical leads 150 a-e. While FIG. 8 shows an impedance sensor array 200 having five (5) bonding pads 140, it will be appreciated that the impedance sensor array 200 can have 2, 3, 4, 5, 6, 7, 8, 9, 10, 11, 12, 13, 14, 15, 16, 17, 18, 19, 20 or more bonding pads 140. In some embodiments, the impedance sensors can all be coupled to a common ground 210 as previously described with respect to FIG. 5. In further embodiments, the electrical conductivity sensor 100 having an impedance sensors 110 with interdigitated electrodes 300 can further contain a common counter electrode 230, which can be configured as shown and described with respect to FIG. 7 The dimensions of these embodiments of the electrical conductivity sensor 100 can be as described with respect to FIGS. 1-3 above. The electrical conductivity sensor 100 and components thereof can be made from suitable materials as previously described with respect to FIGS. 1-3.

In some embodiments, the electrical conductivity sensor 100 as described in relation to any of FIGS. 1-8 can further contain one or more additional sensors to measure additional tissue characteristics. Additional sensors include, but are not limited to, pH sensors, temperature sensors, chemical sensors, and gas (e.g. CO.sub.2, NO, O.sub.2) sensors. There can be 1, 2, 3, 4, 5, 6, 7, 8, 9, 10 or more additional sensors. The additional sensors can also be configured as an array akin to the impedance sensor array on the electrical conductivity sensor 100. The additional sensor(s) can be coupled to the substrate 160. The additional sensors can be coupled to one or more additional bonding pads via leads as will be appreciated by those of skill in the art.

The electrical conductivity sensor 100 and/or any component(s) thereof as described in relation to any of FIGS. 1-8 can be disposable, reusable, recyclable, biocompatible, sterile, and/or sterilizable.

The electrical conductivity sensor 100 and components thereof described herein can be manufactured by any suitable method and in any suitable way. Suitable methods include, but are not limited to, injection molding, 3-D printing, glass/plastic molding processes, optical fiber production process, casting, chemical deposition, electrospinning, machining, die casting, evaporative-pattern casting, resin casting, sand casting, shell molding, vacuum molding, thermoforming, laminating, dip molding, embossing, drawing, stamping, electroforming, laser cutting, welding, soldering, sintering, bonding, composite material winding, direct metal laser sintering, fused deposition molding, photolithography, spinning, metal evaporation, chemical etching and sterolithography. Other techniques will be appreciated by those of skill in the art.

### Electrical Conductivity Probes

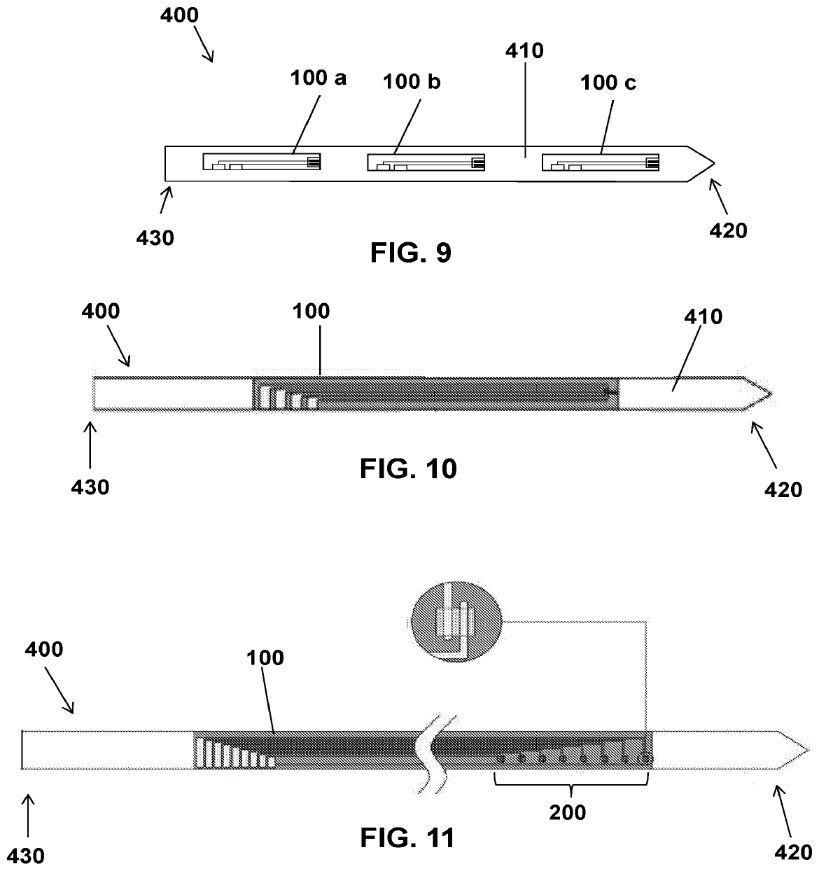

The electrical conductivity sensors 100 described in relation to FIGS. 1-8 can be coupled to or integrated with a probe. In some embodiments, the probe can be a treatment probe (i.e. the probe delivering the focal ablation/cell membrane disruption therapy). The probe that contains the electrical conductivity sensor 100 can be separate from the treatment probe. With the general concept in mind, attention is directed to FIGS. 9-11, which show various embodiments of probes including electrical conductivity sensors 100 as described in relation to FIGS. 1-8. As shown in FIGS. 9-11, which show embodiments of an electrical conductivity probe 400 having one or more electrical conductivity sensor 100 a,b,c (collectively 100). The electrical conductivity sensor(s) 100 can be any electrical conductivity sensor described in relation to FIGS. 1-8.

The electrical conductivity probe 400 can have an elongated member 410 having a distal portion 420 and a proximal portion 430. The elongated member 400 can be any three dimensional shape, including but not limited to, an irregular shape, a cylinder, a cannula, a cuboid, and a triangular prism. The elongated member 400 can have a width. The width can range from about 0.1 mm to about 10 mm or more. The elongated member can have a length. The length can range from about 5 mm to about 50 cm or more. The elongated member can have a diameter. The diameter can range from about 10 microns to about 10 mm or more. The distal portion can have a tapered, beveled, pointed, blunt, sharp, rounded, or flat end. Other configurations for the elongated member will be appreciated by those of skill in the ar. At least one electrical conductivity sensor 100 a,b,c (collectively 100) coupled to or otherwise integrated with an outer surface of the elongated member. In some embodiments, 1, 2, 3, 4, 5, 6, 7, 8, 9, 10 or more electrical conductivity sensors 100 can be coupled to the elongated member 410. In some embodiments the electrical conductivity sensor(s) 100 can be removably coupled to the elongated member 410. The electrical conductivity sensor(s) 100 can be electrically coupled to the elongated member 410. The electrical conductivity sensor(s) 100 can be coupled to the elongated member in any desired configuration, e.g. linearly, radially, and the like, as will be appreciated by those of skill in the art.

The electrical conductivity probe 400 can include sensors configured to detect tissue characteristics (e.g. pH, temperature, chemical, gas sensors) and circuitry as needed. In some embodiments, the electrical conductivity probe 400 can be configured to deliver an energy to result in focal ablation/cell membrane disruption in a tissue. Stated differently, the electrical conductivity probe 400 can also be a treatment probe in some embodiments. In other embodiments, the electrical conductivity probe 400 can be separate from a treatment probe. The electrical conductivity probe 400 and/or components thereof can be disposable, reusable, recyclable, biocompatible, sterile, and/or sterilizable.

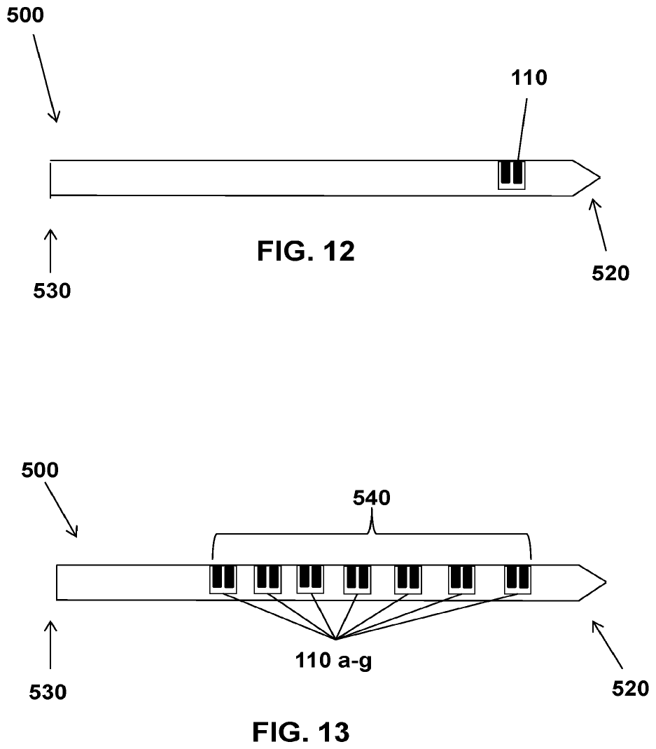

In some embodiments, the impedance sensors and impedance sensor arrays can be integrated directly with an elongated member 510 of an electrical conductivity probe 500. In other words, the impedance sensor and impedance sensor arrays and associated circuitry are not coupled to a substrate (e.g. 160, FIGS. 1-8), but rather directly integrated with an elongated member 510 of a probe. With this in mind attention is directed to FIGS. 12-13, which show embodiments of an electrical conductivity probe 500 having one (FIG. 12) or more (FIG. 13) impedance sensors 110, which can be configured to measure tissue impedance, a change in tissue impedance across regions of tissue, and/or both low-frequency (a region) impedance and high-frequency (β region) impedance. The impedance sensor(s) can be as described in relation to any of FIGS. 1-8. As shown in FIG. 13, the impedance sensor(s) can be positioned on the elongated member such that they can form an impedance sensor array 540. The elongated member can be as described in relation to FIGS. 9-11.

The impedance sensor(s) 110 can be electrically coupled to the elongated member 410. The electrical conductivity probe 500 can include additional sensors (e.g. pH, temperature, chemical, gas sensors) and additional circuitry as needed. In some embodiments, the electrical conductivity probe 500 can be configured to deliver an energy to result in focal ablation/cell membrane disruption in a tissue. Stated differently, the electrical conductivity probe 500 can also be a treatment probe in some embodiments. In other embodiments, the electrical conductivity probe 500 can be separate from a treatment probe. The electrical conductivity probe 500 and/or components thereof can be disposable, reusable, recyclable, biocompatible, sterile, and/or sterilizable.

The electrical conductivity probes 400, 500 described herein can be manufactured by any suitable method and in any suitable way. Suitable methods include, but are not limited to, injection molding, 3-D printing, glass/plastic molding processes, optical fiber production process, casting, chemical deposition, electrospinning, machining, die casting, evaporative-pattern casting, resin casting, sand casting, shell molding, vacuum molding, thermoforming, laminating, dip molding, embossing, drawing, stamping, electroforming, laser cutting, welding, soldering, sintering, bonding, composite material winding, direct metal laser sintering, fused deposition molding, photolithography, spinning, metal evaporation, chemical etching and sterolithography. Other techniques will be appreciated by those of skill in the art.

### Real-Time Lesion/Treated Area Monitoring Systems

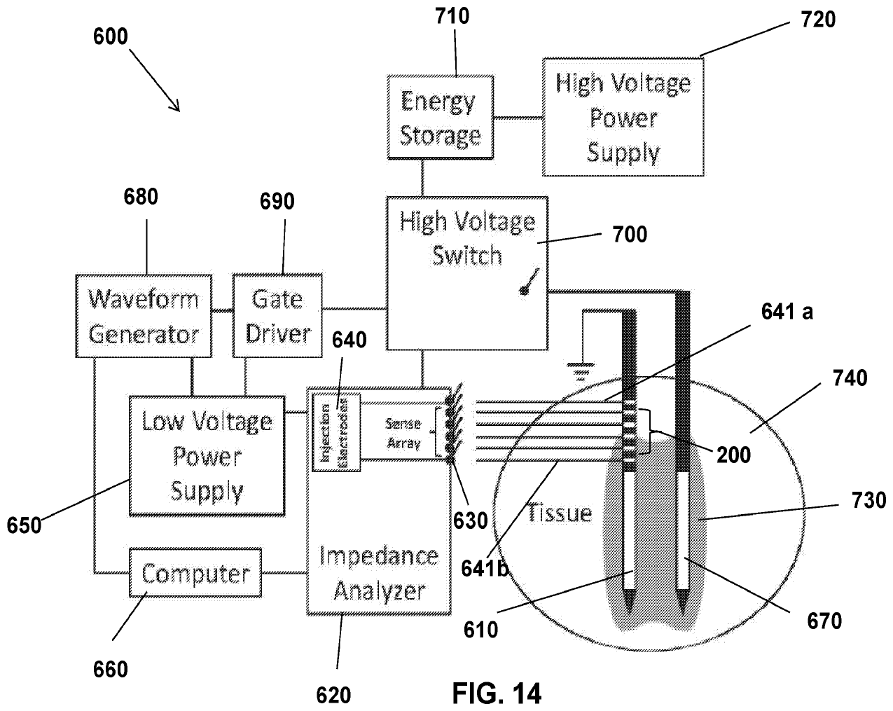

Also provided herein are lesion and treated area monitoring systems that can include one or more electrical conductivity probes and components thereof described in relation to FIGS. 1-13 that can monitor lesion formation during focal ablation/cell membrane disruption therapy. Discussion of the various systems begins with FIG. 14, which shows embodiments of a real-time lesion monitoring system 600. An electrical conductivity probe 610 can be coupled to an impedance analyzer 620. The electrical conductivity probe 610 can be any electrical conductivity probe as described in relation to FIGS. 9-13. The impedance analyzer 620 can be electrically coupled to the impedance sensor(s) 110 of the electrical conductivity probe 610. In some embodiments, the impedance analyzer can contain one or more switches 630, where each switch can be coupled to a single impedance sensor on the electrical conductivity probe 610.

The impedance analyzer 620 can include or be coupled to one or more current injection electrodes 640 configured to inject a low voltage (0.1-1000 mV or more) signal into the impedance sensor(s) 110 of the electrical conductivity probe 610. The injection electrode(s) 640 can each be coupled to an impedance sensor 110 via a switch. Not all of the impedance sensors need be coupled to an injection electrode 640. Stated differently, in some embodiments, only some of the impedance sensors are coupled to an injection electrode via a switch. In some embodiments, the injection electrodes 641 a,b are separate from the impedance sensor(s) 110 and can be placed on the outside of an impedance sensor array 200. (see e.g. FIG. 14). The impedance analyzer and/or injection electrodes can be coupled to a low voltage power supply 650.

The impedance analyzer 620 can be coupled to and/or in communication with a computer or other data storage/processing device 660. The impedance analyzer 620 can be wirelessly coupled to the computer 660. The impedance analyzer can be hard wired to the computer 660. The computer 660 can contain processing logic configured to analyze data from the impedance analyzer 620 or other sensor information received from the electrical conductivity probe 610 and determine the size of the lesion or treated area 730 in the tissue 740. The computer 660 can contain processing logic configured to generate or initiate a signal (visual, audible, digital or otherwise) to alert a user that the lesion or treated are has reached a threshold size. The computer 660 can contain processing logic that can be configured to analyze data received from the impedance analyzer 620 and/or electrical conductivity probe 610 can contain processing logic configured to analyze data from the impedance analyzer 620 or other sensor information received from the electrical conductivity probe 610 and generate an electrical tomographic image of the treatment area. In some embodiments, the processing logic can be configured to determine the ratio of low-frequency impedance to high frequency impedance at a given impedance sensor 110 from impedance sensor data received from the impedance analyzer 620 and/or electrical conductivity probe 610. The computer 660 can contain processing logic configured to determine the amount of high voltage that should be applied to the treatment area via a treatment probe 670 in response to the impedance data and/or other sensory information received.

The computer 660 can be coupled to a waveform generator 680. The waveform generator 680 can be coupled to a gate driver 690. The gate driver 690 and/or impedance analyzer 620 can be coupled to a high voltage switch 700. The high voltage switch can be coupled to an energy storage device 710. The energy storage device can be coupled to a high voltage power supply 720, configured to deliver a high voltage that can range from 50 to 10000 V or more. A treatment probe 670 can be coupled to the high voltage switch 700. The high voltage switch 700 can be controlled by and/or responsive to the waveform generator 680 and/or gate driver 690. Insofar as the waveform generator 680 and/or gate driver 690 can be controlled by the computer 660, treatment can be, in some embodiments, autonomously controlled in response to impedance and other sensory data obtained by the electrical conductivity probe 610 during treatment. The operation of the system is discussed in further detail below.

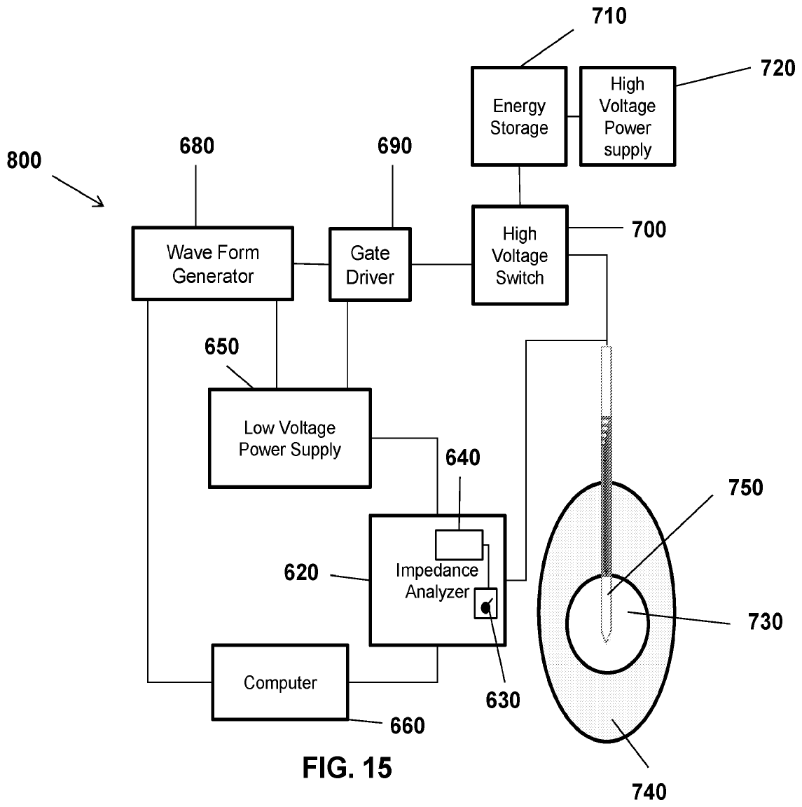

In some embodiments, such as those shown in FIG. 15, the electrical conductivity sensor only includes one sensing area as opposed to an array of sensors which provides case of fabrication and could be used to tell if the lesion front has reached a certain point rather than monitoring its location. The system 800 can be configured the same as that described in relation to FIG. 14, except that a single probe 750, which can contain one or more impedance sensor or an impedance sensor array, is coupled to both the high voltage switch 700 and the low voltage power supply 650.

### Real-Time Lesion Front/Treated Area Monitoring

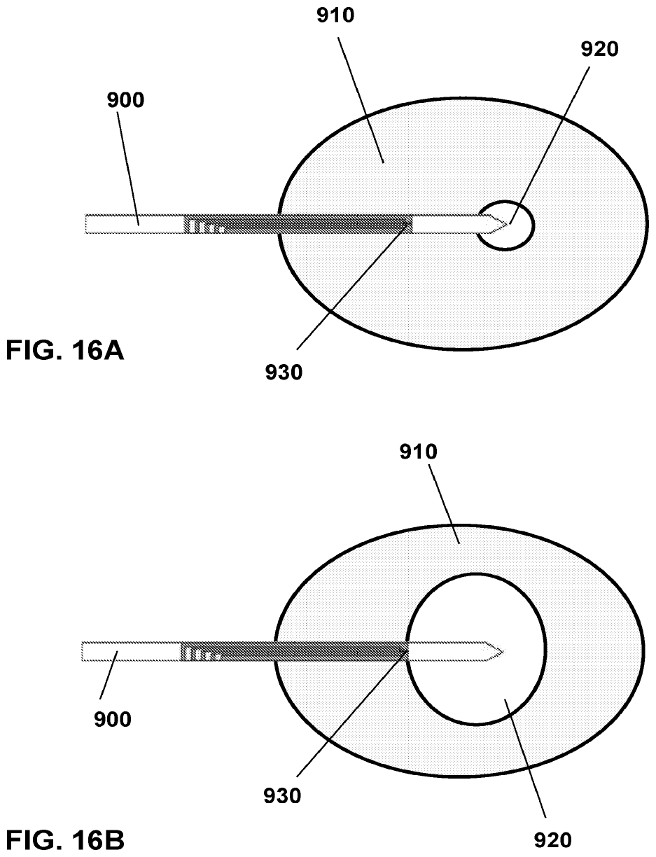

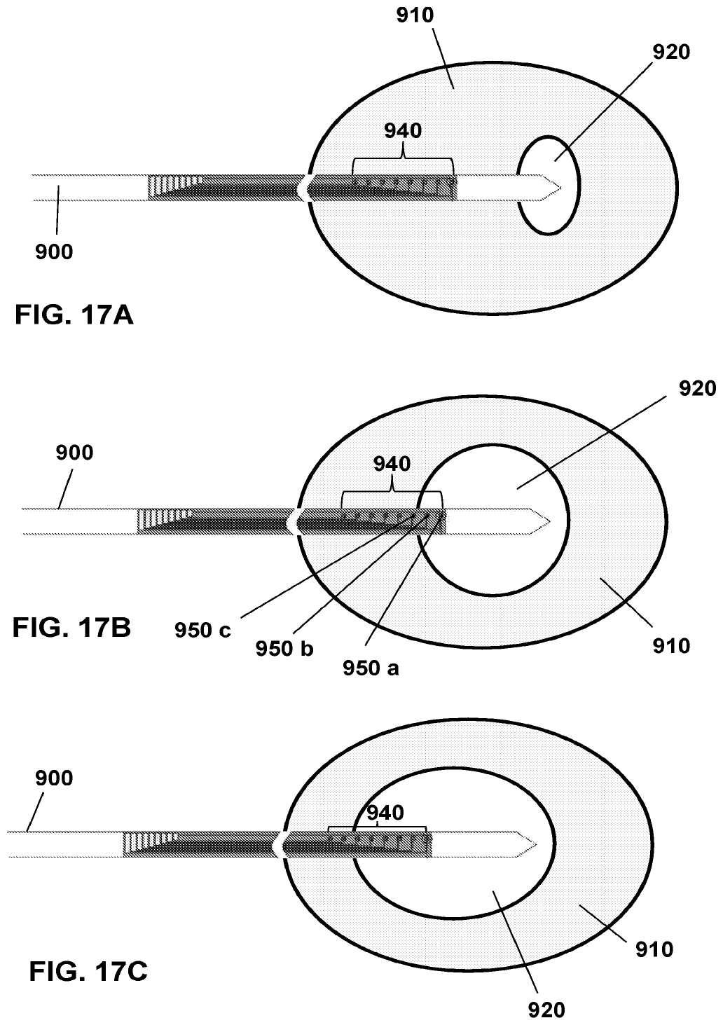

The devices and systems described herein can be used to monitor the lesion formation/front and/or treated area during focal ablation/cell membrane disruption therapies, which include, but are not limited to radiofrequency (RF) ablation, microwave ablation, laser ablation, cryo-ablation, ultrasound, electroporation (reversible and irreversible), supraporation, and radiation therapy. Thus, these devices and systems have application for tumor and undesired ablation, drug delivery, and gene therapy and nucleic acid and other molecule delivery. In principle, an electrical conductivity probe as described in relation to FIGS. 1-13 can be inserted into a tissue. During focal ablation or cell membrane disruption, the treated portion of the tissue undergoes changes due to changes in the permeability of the cell membrane. This results in the formation of a lesion or treated area (e.g. area of tissue to which a drug or other molecule has been delivered). As treatment continues the size of the lesion or treated area can grow. Impedance and other sensors on the electrical conductivity probe can measure electrical conductivity, pH, temperature, chemicals, and/or gasses at locations in the tissue. The systems and devices described herein can then determine the lesion size based upon the electrical conductivity data and other sensory information determined by the probe. In some embodiments, the system can be configured to autonomously control the treatment probe such that when the lesion has reach a desired size, the system can stop treatment in the tissue. In embodiments, the system can be configured to alert a user that the lesion/treated are has reached a desired size. In some embodiments, a user can alter treatment in response to the determined lesion/treated area size. The operation of the systems and devices is discussed in greater detail with respect to FIGS. 16A-17C.

Discussion of the operation of the systems and devices begins with FIGS. 16A-16B, which show monitoring of a lesion/treated area formation and front using an electrical conductivity probe having an impedance sensor during treatment (FIG. 16A) and at the treatment endpoint (FIG. 16B). The treatment probe is not shown in FIGS. 16A and 16B for clarity. However, it will be appreciated that treatment may be provided by a separate treatment probe or be provided by the electrical conductivity probe, which can be configured to deliver high voltage treatment as well as measure tissue characteristics. FIGS. 16A-16B demonstrate monitoring of lesion/treated area formation and front during treatment when using a single impedance sensor (or other sensor) or multiple impedance sensors (or other sensors) placed radially about the surface of the probe such that the sensors are all at the same point along the length of the probe.

As shown in FIG. 16A, the electrical conductivity probe 900 can be inserted into the tissue 910. The electrical conductivity probe 900 can be inserted into the tissue such that the impedance or other sensor is at the outer edge of the desired treatment area. As treatment begins, a lesion or treated area 920 begins to form as the permeability of the cell membranes change. During this time impedance and/or other tissue characteristics are being measured by the sensor(s) 930 on the electrical conductivity probe. The sensors (impedance or other types) can be as described in relation to FIGS. 1-8. As such, during treatment, the impedance and/or other tissue characteristics can be continually determined during treatment and compared to prior measurements, including any baseline measurements taken prior to the start of treatment, to determine if the lesion/treated area has reached the desired size. As shown in FIG. 16B, when the lesion/treated area has grown such that it reaches the point in the tissue where the impedance or other sensor(s) 930 is located, the sensor(s) will measure a change in electrical conductivity and/or pH, chemical concentration, gas concentration, or other molecule concentration and the system can alert a user that the size of the lesion/treated area has reached the desired size. For example, in some embodiments, when the lesion/treated area reaches the sensor(s) 930 on the electrical conductivity probe 900, the low-frequency impedance is equal to the high-frequency impedance. In other embodiments, the system can automatically stop treatment via the treatment probe in response to a detected change in the impedance or other tissue characteristic.

While systems and devices employing sensor(s) at a single point along the length of the probe can be suitable for some applications, they can only determine the size of a lesion/treated area when it reaches a single point. With that in mind attention is directed to FIGS. 17A-17C, which show the operation of an electrical conductivity probe having a sensor array (e.g. an electrical impedance sensor array) during treatment. The treatment probe is not shown in FIGS. 17A-17B for clarity. However, it will be appreciated that treatment may be provided by a separate treatment probe or be provided by the electrical conductivity probe, which can be configured to deliver high voltage treatment as well as measure tissue characteristics.

As shown in FIG. 17A, the electrical conductivity probe 900 can be inserted in a tissue 910 to be treated. Baseline impedance and other tissue characteristic measurements can be obtained prior to the start of treatment. As treatment begins a lesion/treated area 920 can form in the tissue 910. During treatment the sensors of the sensor array 940 can be measuring impedance and/or other tissue characteristics (e.g. pH, chemical concentration, gas concentration, temperature, other molecule concentration, and the system (not shown for clarity) can be determining if there is a change in the impedance and/or other tissue characteristics at any given sensor along the sensor array 940 or between any combination of sensors along the sensor array 940. As the lesion front/treatment area 920 grows (see FIG. 17B), the system will determine that there is a change relative to base line and/or that of another sensor in the impedance and/or other tissue characteristic between certain sensors within the array. From that data the system can determine the size of the lesion and/or determine the position of the lesion front as the lesion grows during treatment. For example, as shown in FIG. 17B the lesion/treated area 920 has grown such that the lesion front is between the second 950b and third sensor 950c of the sensor array 940. As such, the system can determine that there is a change in the impedance (or other tissue characteristic) at the second sensor 950b relative to baseline. The system can determine that there is no change in the impedance (or other tissue characteristic) at the third sensor 950c relative to baseline. From this, the system can determine that the lesion front/treated area has reached the position on the probe that lies between the second 950b and third 950c sensor on the electrical conductivity probe 900. The process of continually measuring impedance (other tissue characteristic) by the sensors of the sensor array 940 and comparing them to baseline/and or other data from other sensors of the sensor array 940 can continue until the lesion/treated area 920 has reached a desired size. The desired size can be predetermined and the system can be configured to alert a user via a signal when the system calculates that the desired size has been reached. In other embodiments, the system can be configured to automatically stop treatment when the system calculates that the desired size has been reached.

It will be appreciated that any number of electrical conductivity probes 900 can be used at the same time. By placing electrical conductivity probes 900 at different locations and depths into the tissue, the data provided can be used by the system to determine a volume of the lesion/treated area and/or generate a three dimensional image of the treated area.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

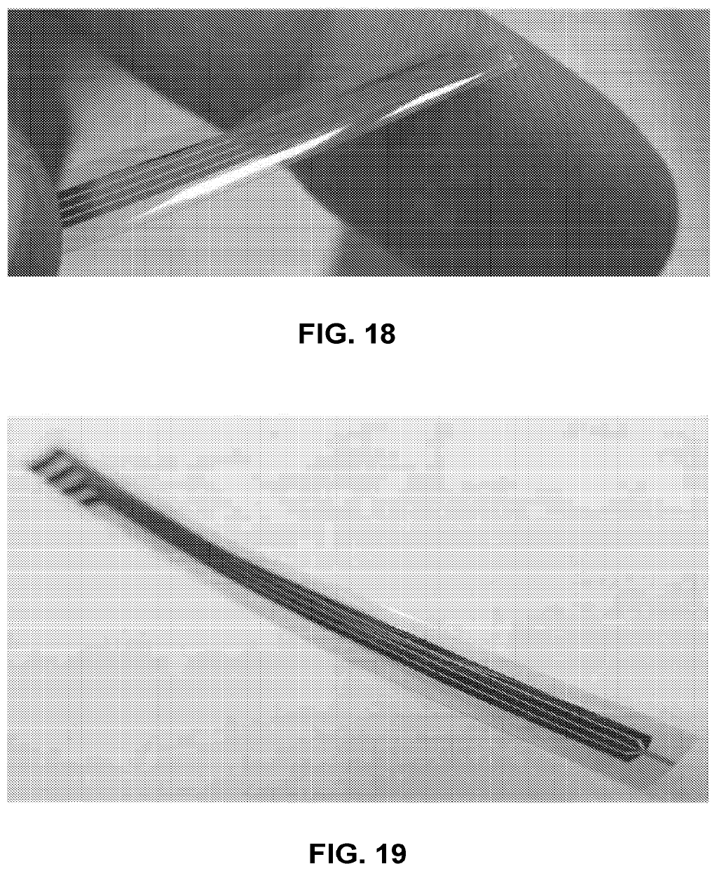

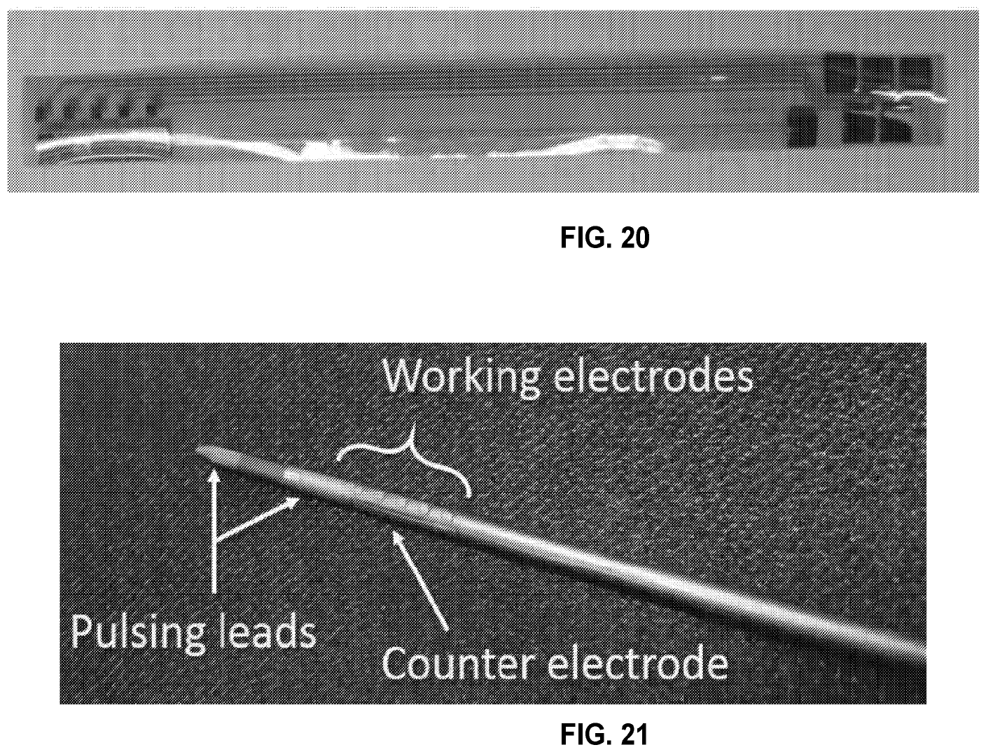

FIGS. 18-20 show images demonstrating an electrical conductivity sensor as described in relation to any of FIGS. 1-8. The fabricated probe is about 15 micron thick, 8 cm long and 8 mm wide. The gold wires are sandwiched between two polyimide layers. The polyimide layer over the bonding pads and the sensor area is removed to expose these parts. The small dimensions of the sensor can enable conductivity measurement with a high spatial resolution. The electrical conductivity sensor can be wrapped around a probe, such as an irreversible electroporation probe (IRE) or other treatment probe to create a device capable of both treating tissue with electroporation and monitoring the extent of the treatment in real-time. In this probe, the conductivity measurement can be conducted at one point next to the beginning of the exposed area of the IRE probe. The electrode can be flexible enough to be easily wrapped around IRE probes with a small diameter of 1 mm. FIG. 21 shows an image of an electrical conductivity sensor that has been coupled to an IRE probe.

### Example 2

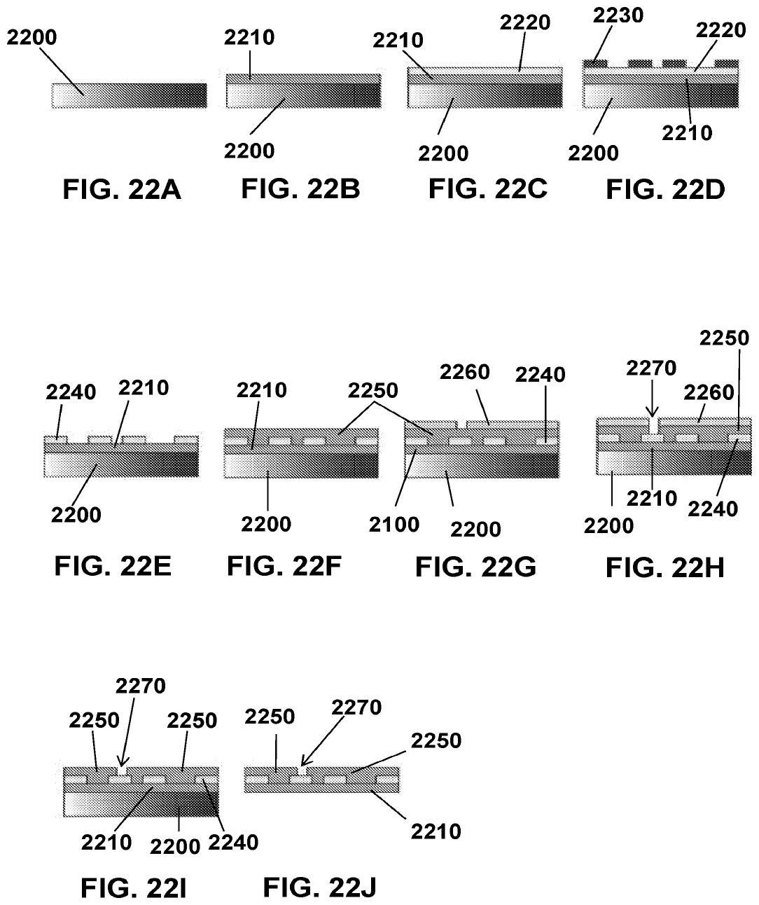

FIGS. 22A-22J demonstrate a fabrication process for the construction of the electrical conductivity sensor of Example 1. A 4″ Si wafer 2200 was used as the fabrication substrate. The wafer edge was treated with a solution of adhesion promoter (HD Microsystems, Parlin, N.J.) to provide adhesion between the wafer 2200 and the polyimide layer 2210 (FIG. 22B). The adhesion should be enough to keep the construct on the wafer 2200 during the fabrication steps. (FIG. 22B) Polyimide (HD Microsystems, Parlin, N.J.) substrate 2210 was spun and cured over the Si wafer 2200. The spin speed was adjusted to achieve a thickness of about 15 microns. The spin step could be repeated if a greater film thickness is desired. (FIG. 22C) A layer of about 300 nm of gold was deposited on the polyimide layer by PVD. For a better adhesion of gold to the polyimide substrate a Cr layer 2200 was deposited first. (FIG. 22C) A photoresist layer 2230 was spun and patterned as the desired gold electrodes using the photolithography techniques. FIG. 22D The patterned photoresist was used as a mask for wet etching of gold in the next step. Gold and Cr layers were etched 2240 (FIG. 22E) using appropriate wet etching solutions and the photoresist layer 2230 was washed away. Another layer of polyimide 2250 was spun and cured to act as an insulator over the electrode FIG. 22F. The insulator should cover the wires of the electrode and leave the sensor and bond pads exposed. A Ti mask 2260 was deposited by PVD and patterned by photolithography techniques followed by wet etching (FIG. 22G). The Ti mask was used to etch the upper polyimide layer in RIE (Reactive Ion Etching) to expose 2270 the sensing areas and bond pads (FIG. 22H). The Ti mask was washed away using wet etching (FIG. 22I). The whole electrode structure was peeled off the Si wafer 2200 (FIG. 22J). To protect the impedance electrodes from high voltage electric discharge of the pulsing leads, a thin passivation layer such as silicon dioxide or silicon nitride can be coated on the sensor area. This passivation layer acts as a capacitor which protects the sensor from high voltage of the DC pulses however has a minimal impact on the AC impedance readings. Functionalization of the sensors with receptor molecules configured to specifically bind a target molecule can performed after metal patterning as an option using techniques known in the art.

### Example 3

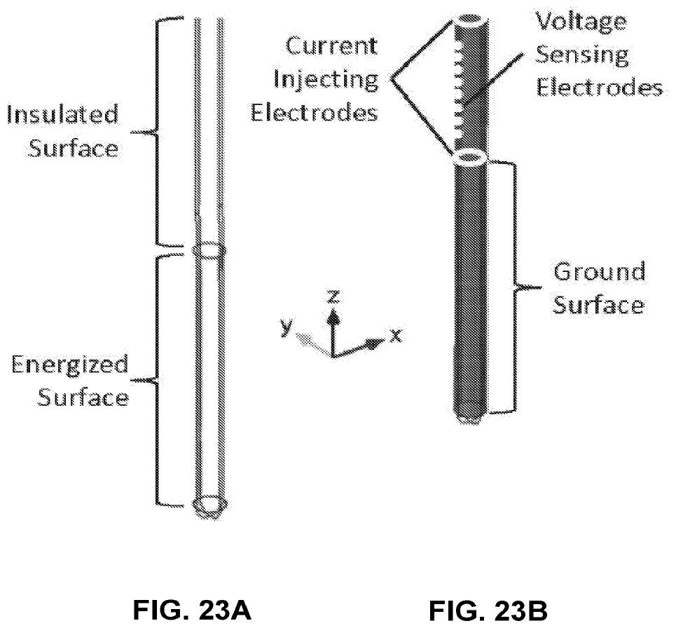

A three dimensional finite element model was constructed in Comsol 4.2a (Burlington, Mass.) to simulate IRE treatment of liver tissue with two needle electrodes (FIGS. 23A-23B). The electric potential distribution within the tissue was obtained by transiently solving:

$$0 = - ∇ \cdot ( σ ( \vert E \vert ) ∇ ϕ ) \qquad ( Equation 1 )$$

Where ϕ is the electric potential, E is the electric field, and a is the electric conductivity. Equation 1 is obtained from Maxwell's equations assuming no external current density (J=σE), no remnant displacement (D=ε₀εᵣ₁E), and the quasi-static approximation. This approximation implies a negligible coupling between the electric and magnetic fields (∇×E=0), which allows for the expression of electric field only in terms of electric potential:

$$E = - ∇ ϕ \qquad ( Equation 2 )$$

As depicted in Equation 1, the electric conductivity is a function of the electric field magnitude. This equation is used to describe the nonlinear of effects of pore formation in the cell membrane at the tissue scale. Specifically, this can be described by a step function with a certain degree of smoothing, or by other functions that follow similar relationships between the electric conductivity and electric field, such as sigmoid or Gompertz functions. The step function chosen here increased from a baseline conductivity of 0.3 S/m to a plateau of 1.05 S/m across a transition zone of 500 V/cm centered at 500 V/cm. Therefore, regions of tissue subject to an electric field above 750 V/cm were maximally electroporated.

An electric potential boundary condition of 1500 V was applied along the energized surface of one of the electrodes, with the corresponding ground portion of the alternate electrode set to 0 V. The dielectric properties of the exposed portion of the electrodes for performing IRE and the insulative portion for protecting healthy tissue can be found in Garcia, P. A., et al., Intracranial Nonthermal Irreversible Electroporation: In Vivo Analysis. Journal of Membrane Biology, 2010. 236(1): 127-136. All remaining interior boundaries were treated as continuity, and all remaining outer boundary conditions were treated as electrical insulation. The stationary problem consisting of 100,497 mesh elements was solved using an iterative, conjugate gradient solver.

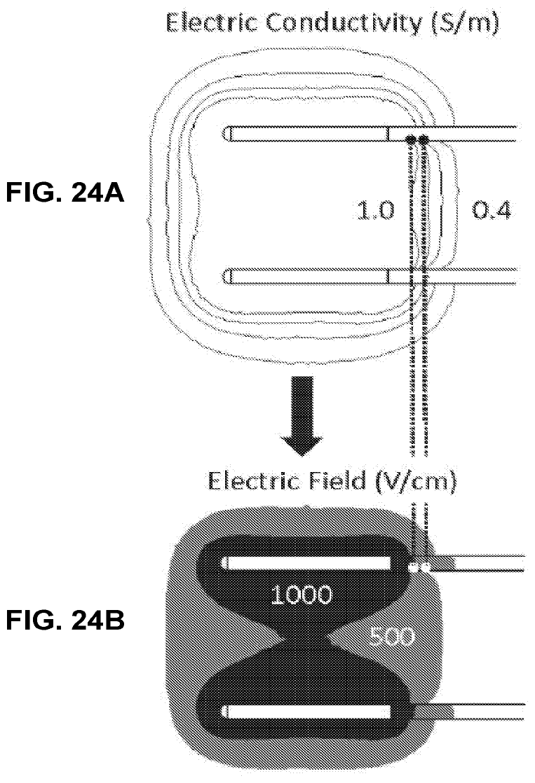

The electrical conductivity in the tissue resulting from IRE is shown in FIG. 24A. Experimentally, voltage drop measurements made between any combination of sensing electrodes can be used to determine this conductivity. Through comparisons to electrical measurements made prior to treatment, it is then possible to determine the extent to which tissue adjacent to each of the sensors has undergone electroporation. If impedance measurements are obtained between electroporative pulses of a multiple pulse protocol, then a real-time, dynamic representation of how the treated tissue expands along the length of the electrode can be obtained. Point specific measurements can also be extrapolated in three dimensions to determine the spatial-temporal conductivity map and electric field distribution (FIG. 24B).

### Example 4

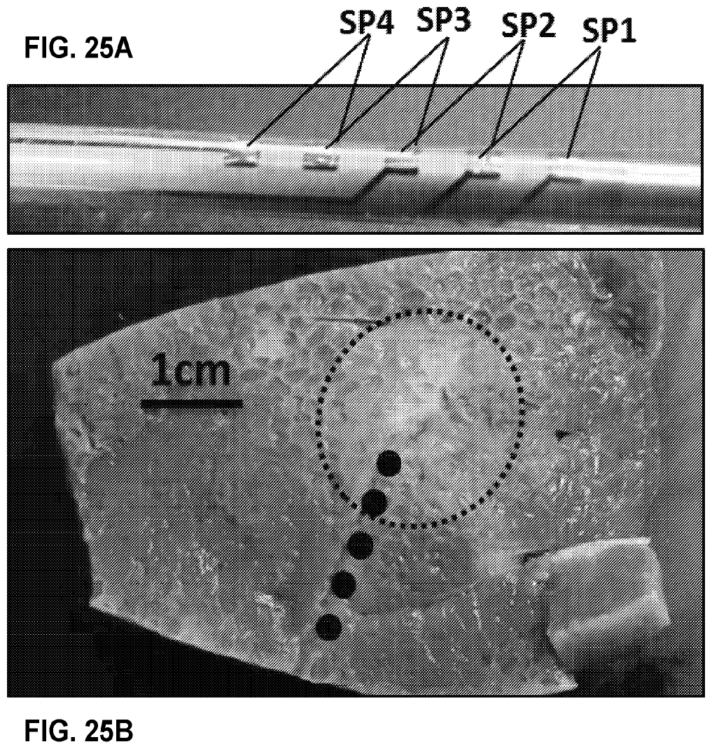

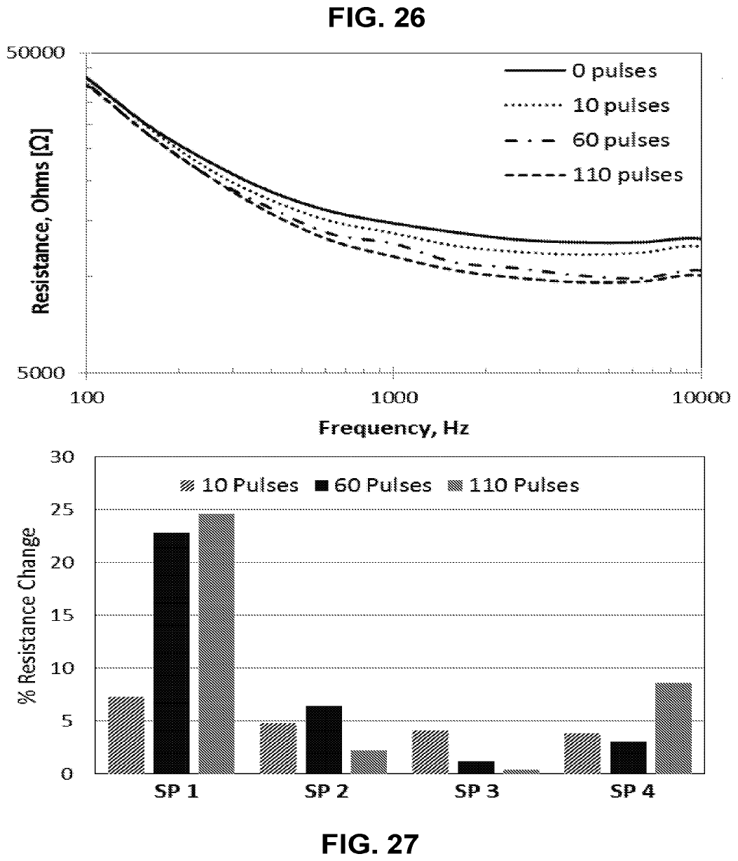

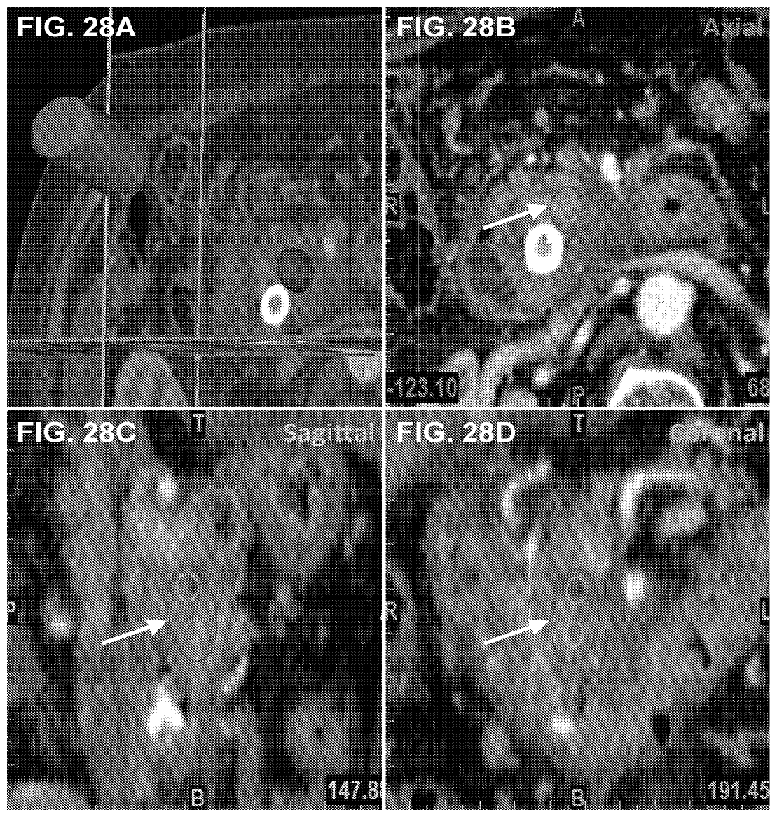

FIGS. 25A-27 describe results of delivering a series of high-frequency irreversible electroporation (HFIRE) pulses to porcine liver through the high voltage portion of a probe that also contains an impedance sensor array. FIG. 25A shows an experimental probe model with 5 microelectrodes and 4 sensing pairs (SP). In FIG. 25B, TTC Stained HFIRE ablation in liver (2000 V) can be observed in which viable tissue was stained red while dead tissue whitened. Ablation (marked by dotted line) reached only SP1. The impedance signature throughout delivery of HFIRE pulses as measured by SP1 is shown in FIG. 26). The largest change in impedance was observed at 5 kHz, which indicated current was no longer confined to extracellular pathways and its flowing through the cell membrane—indicating electroporation of tissue. This progressive decline in resistance can be used to monitor ablation growth throughout the therapy. FIG. 27 presents the resulting changes in tissue impedance during HFIRE therapy at 5 kHz. Major changes in impedance were only observed on probe pair in contact with treated tissue (FIG. 25B). FEM results for electric field distribution along the length of the probe for different pulse parameters can be correlated to these spatio-temporal changes in electrical conductivity during IRE procedures to indicate the electric field threshold for cell death in a tissue of interest.

### Example 5

A real-time visualization tool for monitoring of reversible and irreversible electroporation treatments. Once the threshold for cell death in terms of bulk tissue conductivity has been characterized this information can be used to reconstruct the ablation in 3D. The volume of the ablation geometry can be described in 2D with a Cassini oval plot that has the results from one axis extrapolated into a third dimension.

The Cassini oval is a curve that derives its values based on the distance of any given point, a, from the fixed location of two foci, q.sub. 1 and q.sub.2, located at (x.sub.1, y.sub.1) and (x.sub.2, y.sub.2). The equation is similar to that of an ellipse, except that it is based on the product of distances from the foci, rather than the sum. This makes the equation for such an oval:

$$[ {( {x}_{2} - a )}^{2} + {( {y}_{2} - a )}^{2} ] = {b}^{4} \qquad ( Equation 3 )$$

where b⁴ is a scaling factor to determine the value at any given point. For incorporation of this equation into shapes that represent the electric field distribution, it is assumed that the two foci are located at the center of the pulsing electrodes along the length of the probe (e.g., x-axis) at (±x,0).

Here, the parameter a represents the location of the ablation front along the length of an IRE needle. This is used to solve for b giving a complete equation to describe the ablation volume. After the probe is placed, software can record baseline values for impedance along a micro-sensor array. After treatment begins, impedance measurements can be recorded in real-time. The location of the ablation (lesion) front can be determined according to the characteristic conductivity of the tissue of interested after it has been irreversibly electroporated. Finally, this data can be used to calculate the ablation geometry, which can be projected as a 3D isometric view of SMART probe onto ortho-planes from stacked CT images of patient anatomy (FIG. 28A). Similarly, the ablation progression can be observed during treatment at 10 (green), 50 (red), and 100 (blue) pulses in axial (FIG. 28B), sagittal (FIG. 28C), and coronal planes (FIG. 28D). Ultimately this system can provide healthcare professions and other practitioners with real-time feedback of any IRE therapy, by displaying the ablation volume relative to a targeted tumor in medical scans such as MRI, PET, or CT.

### Example 6

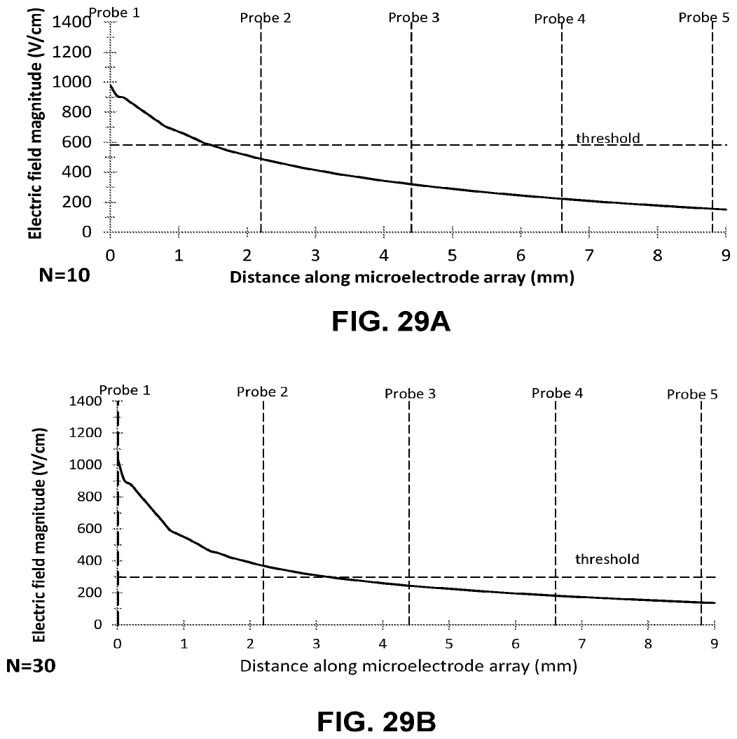

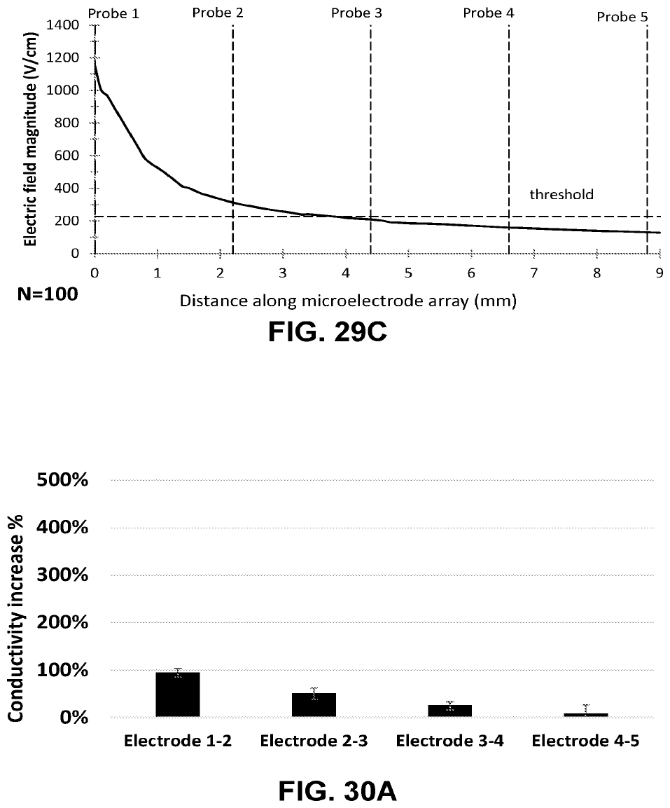

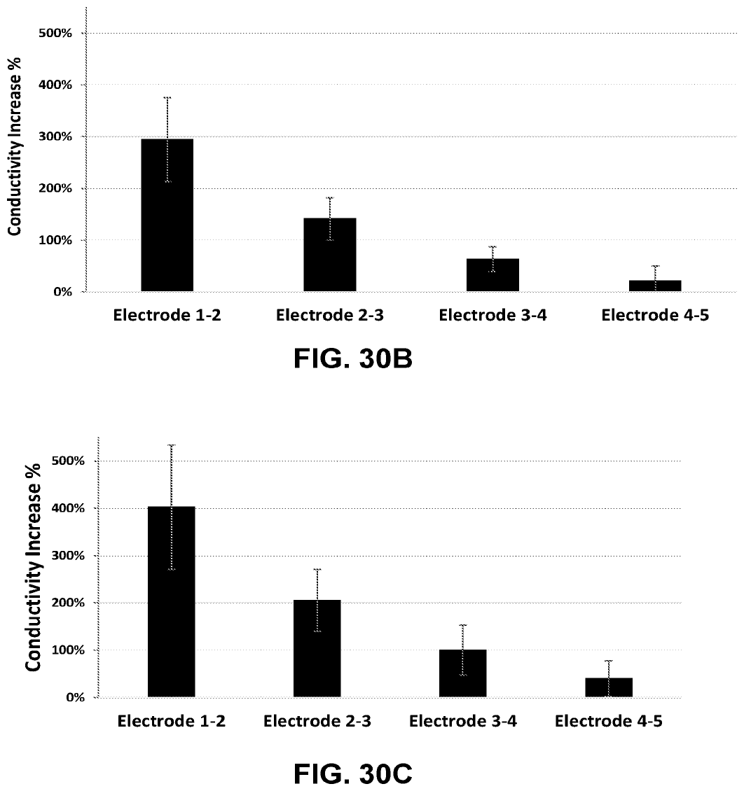

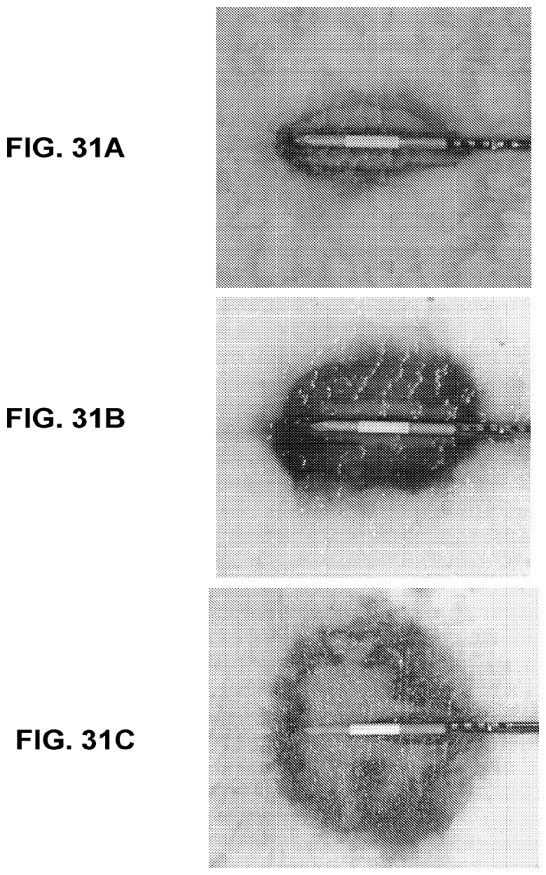

FIGS. 29A-31C describe parts of the methodology related to determining the location of the ablation front and the resulting geometry of the volume of ablation from a series of irreversible electroporation (IRE) pulses through the high voltage portion of a bipolar probe, also containing an impedance sensor array. FIGS. 29A-29C shows the finite element model (FEM) results for electric field distribution along the length of the probe for IRE pulses with a magnitude of 1500V. The dotted line corresponds to a characterized threshold for cell death dependent of a specific number of pulses (N) (e.g., 10, 30, 100). After the tissue has been treated with several IRE pulses an ablation front can be detected in the form of a change in tissue resistivity at different points along the probe (FIGS. 30A-30C). FIGS. 31A-31C shows the resulting volumes of ablation post IRE treatments 10, 30 and 100 pulses of 1500V.

Lesion growth in the perpendicular direction of the probe is also reflected in the impedance measurement by the probe. For example, it is predicted by FEM model (FIGS. 29A-29C, solid line) and observed in FIGS. 31A-31C that for 30 and 100 pulse treatments, probes 1 and 2 would fall within the lesion. However, the corresponding impedance measurement shows 400% and 500% increase in conductivity for 30 and 100 pulses, respectively. This difference is attributed to the depth of lesion in the perpendicular direction. For the case of 10 pulses of 1500V, the small depth of the lesion in perpendicular direction and the marginal location of probe 2 compared with the lesion, results in 200% relative conductivity for sensors 1-2 measurement. For all treatments, the measurements showing 100% relative conductivity correspond to electrodes completely outside of the lesion.

These experimental results show that device (electroporation leads and micro-electrode array) used during these experiments is not only capable of monitoring the lesion length along the probe, but also gives relevant information regarding its other dimensions. This information when combined with FEM modeling can give accurate shape and size of the lesion.

### Example 7

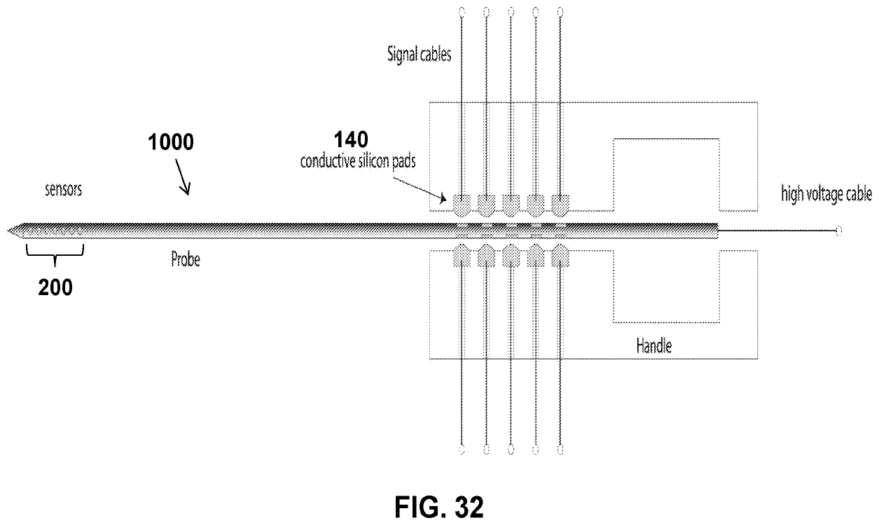

FIG. 32 shows a diagram demonstrating how the electrical connections to a conductivity probe 1000 can be made through conductive flexible silicon pads or any other flexible conductive material or structure that can be installed in the handle and in opposite side of the conductive pads 140. The conductive silicon pads can be connected to the external wires. Upon assembly, the conductive silicon pads come in conformal contact with the gold pads on the conductivity sensor and make the electrical connection.

### Example 8

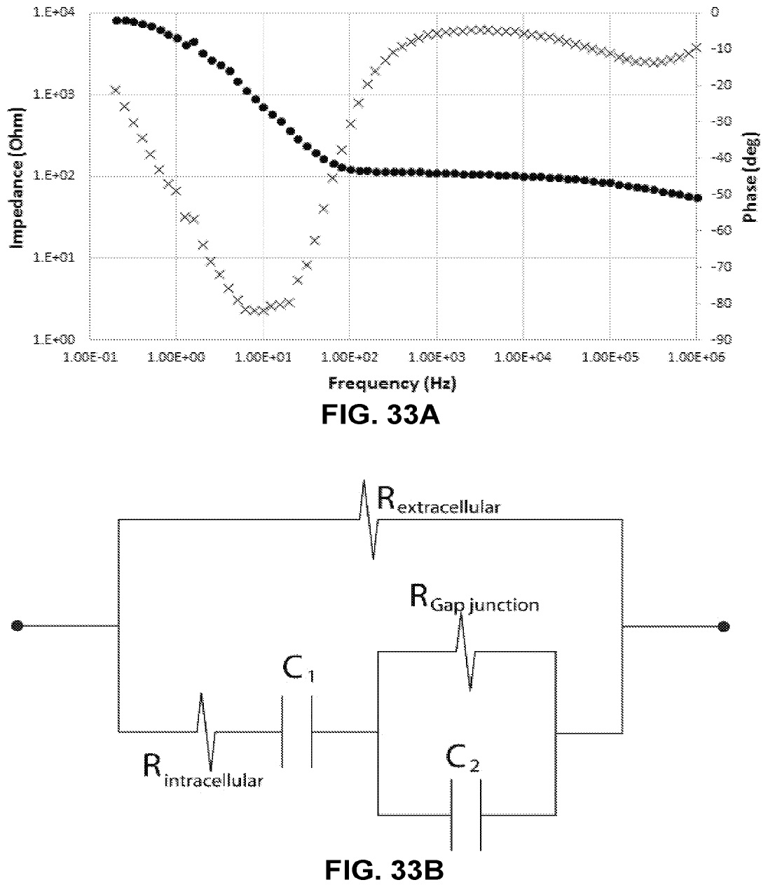

FIG. 33A shows a graph demonstrating the impedance spectrum of porcine liver as measured by the conductivity sensor. Fitting of the spectrum to the equivalent circuit model of tissue reveals critical tissue properties at cellular level which could be used for determination of lesion size during ablation. FIG. 33B shows one example of tissue electric circuit model.

### Example 9

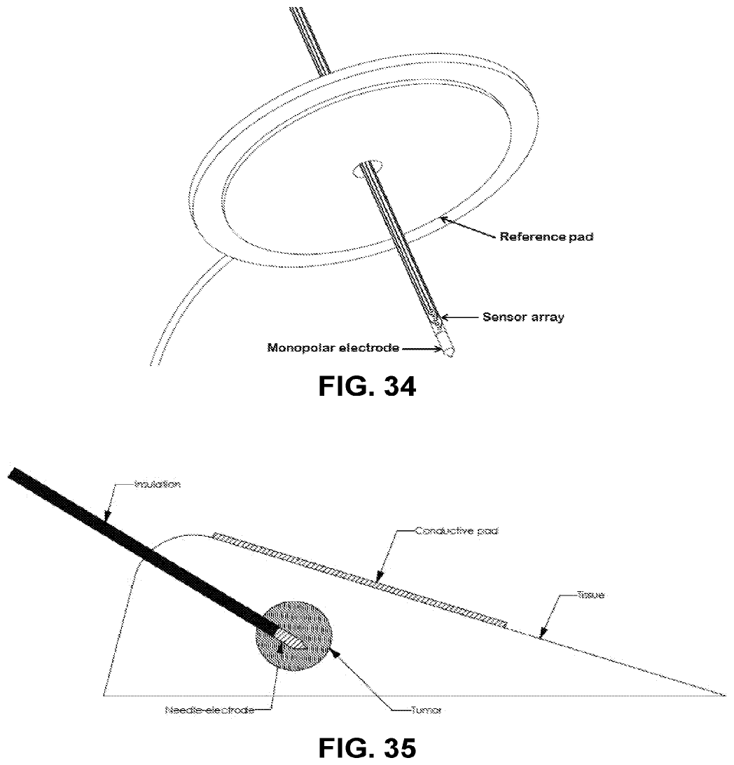

FIGS. 34 and 35 demonstrate additional embodiments of a system configured to monitor a lesion/treated area front in real-time. In this embodiment, the high voltage energy for tissue ablation can be delivered to the tissue through a single high voltage probe and a large grounding pad, which can be positioned on the surface of the organ/tissue. Due to electric field concentration around the tip of the high voltage electrode, a spherical lesion can form. The spherical lesion can be monitored using the conductivity sensor as described before.

1-20. (canceled)

21. A system comprising:
a low voltage power supply;
a high voltage power supply; and
a controller configured to control when a low voltage signal is produced using the low voltage power supply and when a high voltage signal is produced using the high voltage power supply,
wherein the controller is configured to determine if a tissue has been ablated or treated based on one or more low frequency impedance measurements and one or more high frequency impedance measurements.

22. The system of claim 21, further comprising:
an impedance sensor coupled to the low voltage power supply and the high voltage power supply,
wherein the impedance sensor is configured to measure a low frequency impedance generated from the low voltage signal and a high frequency impedance generated from the high voltage signal.

23. The system of claim 22, further comprising:
a probe coupled to the low voltage power supply and the high voltage power supply.

24. The system of claim 23, wherein the probe comprises the impedance sensor.

25. The system of claim 22, wherein the impedance sensor is an impedance sensor array.

26. The system of claim 21, further comprising:
an impedance sensor in communication with the controller,
wherein the controller is configured to control a high voltage treatment signal which is produced by the high voltage power supply based on an impedance measured by the impedance sensor.

27. The system of claim 21, wherein the high voltage power supply is configured to deliver the high voltage signal with a voltage of about 50 V or more.

28. The system of claim 21, wherein the low voltage power supply is configured to deliver the low voltage signal with a voltage of about 0.1 mV or more.

29. The system of claim 21, further comprising:
a treatment probe configured to apply the low voltage signal and the high voltage signal; and
a grounding pad.

30. The system of claim 21, wherein the grounding pad is configured to be placed externally to a tissue.

31. The system of claim 21, wherein the controller is configured to determine a treatment endpoint based on low frequency electrical current measurements.

32. The system of claim 21, further comprising:
an electrical conductivity sensor comprising:
an array of impedance sensors, wherein the array of impedance sensors is configured to measure impedance:
at a first frequency in the range of about 0.001 Hz to 1 kHz; and
at a second frequency in the range of about 0.1 MHz to 100 GHz.

33. The system of claim 32, wherein the electrical conductivity sensor further comprises:
one or more temperature sensors; and
a substrate;
wherein the array of impedance sensors and the one or more temperature sensors are coupled to the substrate.

34. A system comprising:
one or more electrodes;
a waveform generator, wherein the waveform generator is further configured for delivering electrical energy to the one or more electrodes;
a low voltage power supply;
a high voltage power supply; and
a controller operatively coupled to the one or more electrodes, the waveform generator, the low voltage power supply, and the high voltage power supply, wherein the controller is configured to control when a low voltage signal is produced using the low voltage power supply and when a high voltage signal is produced using the high voltage power supply.

35. The system of claim 34, further comprising:
an impedance sensor coupled to the low voltage power supply and the high voltage power supply, wherein the impedance sensor is configured to measure a low frequency impedance generated from the low voltage signal and a high frequency impedance generated from the high voltage signal.

36. The system of claim 35, wherein the controller is configured to control a high voltage treatment signal, which is produced by the high voltage power supply based on an impedance measured by the impedance sensor.

37. The system of claim 34, wherein the high voltage power supply is configured to deliver the high voltage signal with a voltage of about 50 V or more, and wherein the low voltage power supply is configured to deliver the low voltage signal with a voltage of about 0.1 mV or more.

38. The system of claim 34, wherein the one or more electrodes comprise:
a treatment probe configured to apply the low voltage signal and the high voltage signal; and
a grounding pad.

39. The system of claim 34, further comprising:
an electrical conductivity sensor comprising:
an array of impedance sensors, wherein the array of impedance sensors is configured to measure impedance:
at a first frequency in the range of about 0.001 Hz to 1 kHz; and
at a second frequency in the range of about 0.1 MHz to 100 GHz.

40. The system of claim 39, wherein the electrical conductivity sensor further comprises:
one or more temperature sensors; and
a substrate;
wherein the array of impedance sensors and the one or more temperature sensors are coupled to the substrate.
